SEQUENCE LISTING (21 sequences)

SEQ ID NO: 1 - DNA, 59 bp, Artificial Sequence
  ALK_f19-12N
tcgtcggcagcgtcagatgtgtataagagacagcctgatcagccaggaggatacacacg

SEQ ID NO: 2 - DNA, 59 bp, Artificial Sequence
  ALK_f19-4N
tcgtcggcagcgtcagatgtgtataagagacagtgccaagccacagagttggagaagag

SEQ ID NO: 3 - DNA, 57 bp, Artificial Sequence
  ALK_f19-n4N
tcgtcggcagcgtcagatgtgtataagagacagcaagtataaccccacgtgaacgag

SEQ ID NO: 4 - DNA, 62 bp, Artificial Sequence
  B26_i01-2N
tcgtcggcagcgtcagatgtgtataagagacagtggatattgcagcagtcagagcccttaac

SEQ ID NO: 5 - DNA, 61 bp, Artificial Sequence
  BRAF_i15-2N
tcgtcggcagcgtcagatgtgtataagagacaggatccagacaactgttcaaactgatggg

SEQ ID NO: 6 - DNA, 63 bp, Artificial Sequence
  EGFR_i21-2N
tcgtcggcagcgtcagatgtgtataagagacagcctggcagccaggaacgtactggtgaaaac

SEQ ID NO: 7 - DNA, 66 bp, Artificial Sequence
  KRAS_i02-2N
tcgtcggcagcgtcagatgtgtataagagacagggcctgctgaaaatgactgaatataaacttgtg

SEQ ID NO: 8 - DNA, 59 bp, Artificial Sequence
  MET_i14-1N
tcgtcggcagcgtcagatgtgtataagagacagtgcccgaagtgtaagcccaactacag

SEQ ID NO: 9 - DNA, 64 bp, Artificial Sequence
  PIK3CA_i02-1N
tcgtcggcagcgtcagatgtgtataagagacagtctcgattgaggatcttttcttcacggttgc

SEQ ID NO: 10 - DNA, 62 bp, Artificial Sequence
  TP53_i07-2N
tcgtcggcagcgtcagatgtgtataagagacagagtcttccagtgtgatgatggtgaggatg

SEQ ID NO: 11 - DNA, 81 bp, Artificial Sequence
  UA4
  misc_feature (2)..(3): n is a, c, g, or t
  misc_feature (6)..(7): n is a, c, g, or t
  misc_feature (10)..(11): n is a, c, g, or t
  misc_feature (14)..(15): n is a, c, g, or t
gnntgnntgnntgnnctgtctcttatacacatctccgagcccacgagacggaatctcatctcgtatgccgtcttctgcttg

SEQ ID NO: 12 - DNA, 81 bp, Artificial Sequence
  UA5
  misc_feature (2)..(3): n is a, c, g, or t
  misc_feature (6)..(7): n is a, c, g, or t
  misc_feature (10)..(11): n is a, c, g, or t
  misc_feature (14)..(15): n is a, c, g, or t
gnntgnntgnntgnnctgtctcttatacacatctccgagcccacgagacttctgaatatctcgtatgccgtcttctgcttg

SEQ ID NO: 13 - DNA, 56 bp, Artificial Sequence
  i3
aatgatacggcgaccaccgagatctacacaggataggtcgtcggcagcgtcagatg

SEQ ID NO: 14 - DNA, 56 bp, Artificial Sequence
  i4
aatgatacggcgaccaccgagatctacacttatgcgatcgtcggcagcgtcagatg

SEQ ID NO: 15 - DNA, 29 bp, Artificial Sequence
  P5-AMP
aatgatacggcgaccaccgagatctacac

SEQ ID NO: 16 - DNA, 19 bp, Artificial Sequence
  SP-EGFR21-1
tactggtgaaaacaccgca

SEQ ID NO: 17 - DNA, 19 bp, Artificial Sequence
  F4-SP1
tcgtcggcagcgtcagatg

SEQ ID NO: 18 - DNA, 24 bp, Artificial Sequence
  P7-AMP
caagcagaagacggcatacgagat

SEQ ID NO: 19 - DNA, 17 bp, Artificial Sequence
  MGB-iSP2-1
tctcgtgggctcggaga

SEQ ID NO: 20 - DNA, 18 bp, Artificial Sequence
  R-EGFR21
ttccgcacccagcagttt

SEQ ID NO: 21 - DNA, 23 bp, Artificial Sequence
  MGB-EGFR21
tgtcaagatcacagattttgggc

(19) United States
(12) Patent Application Publication
(10) Pub. No.: US 2025/0283155 A1
(43) Pub. Date: Sep. 11, 2025

(54) OPTIMIZED DNA LINEAR AMPLIFICATION METHOD AND TEST KIT

(71) Applicant: APOGENOMICS CO., LTD., SHANGHAI (CN)

(72) Inventor: GUOHUA YANG, SHANGHAI (CN)

(73) Assignee: APOGENOMICS CO., LTD., SHANGHAI (CN)

(21) Appl. No.: 18/272,589

(22) PCT Filed: Feb. 17, 2022
(86) PCT No.: PCT/CN2022/076538

§ 371 Date: Feb. 24, 2024

(30) Foreign Application Priority Data
Jan. 22, 2021 (CN) 202110088796.0

(51) Int. Cl. C12Q 1/6844; C12N 15/10

(52) U.S. Cl. CPC C12Q 1/6844; C12N 15/1093

(57) ABSTRACT

A method for amplifying a DNA target region is provided. The method includes: linearly amplifying a fragmented DNA including the target region by a specific primer to provide a linear amplification product, where 3′ end of the specific primer is modified by a duality functional group, a phosphodiester bond of a part of nucleotide backbone at the 3′ end of the specific primer is modified by phosphorothioate, and the duality functional group is used to prevent the 3′ end of the specific primer from ligation with other oligonucleotides and can be removed by a specific enzyme to perform a linear amplification of the specific primer. The method for amplifying a DNA target region effectively reduces a non-specific amplification during the linear amplification by introducing a phosphorothioate modification into the specific primer.

### TECHNICAL FIELD

The present disclosure relates to the field of biotechnology, in particular to a method for amplifying a DNA target region.

### BACKGROUND

DNA detection is a molecular diagnostic technology for use in multiple fields, such as, infectious diseases, malignant tumors, and prenatal screening. Before DNA detection, it is generally necessary to amplify the target DNA to ensure that the quantity and concentration of the target DNA can reach a detectable level by existing techniques. PCR amplification is currently the mainstream DNA amplification technology. However, the characteristics of PCR exponential amplification (that is, the amplification product of the previous round serves as the template for the next round of amplification) can easily lead to the accumulation of the introduced base errors to affect the detection accuracy; and the rigid condition that the primers are necessary to be designed for both ends also causes restriction to the detection so that the detection of fragmented DNA becomes difficult. Some techniques of DNA linear amplification developed in recent years can fundamentally solve the above-mentioned problems of PCR amplification. Linear amplification refers to a DNA amplification method in which each target region is captured and extended by a single primer, and each round of amplification uses only the DNA molecule in the original sample as a template. The linear amplification is characterized by using only the target DNA as a template in each round, which effectively avoids the accumulation of the introduced base errors; and it is required to design the specific primer for only one end in the linear amplification to achieve the detection, so that the fragmented DNA can be detected more effectively.

However, the efficiency of linear amplification is limited, and tens of cycles are required to achieve an expected amplification factor. Once a non-specific PCR exponential amplification occurs during the amplification, the substrate will be exhausted in the reaction, causing the failure of the linear amplification. There are mainly three reasons for the occurrence of non-specific amplification: 1) Dimers formed between the primers. Since tens to hundreds of primers are used in the multiplex amplification system and have higher concentration thereof than the reaction template, the dimers formed between the primers (that is, the amplification products formed by mis-matching between 3′ ends of the primers) can result in PCR reactions between the primers.

2) Non-specific PCR caused by the primer's off-target. In the case of multiple detection of multiple target DNA sequences, the off-target binding caused by degradation of the primer itself is easy to form a PCR reaction with other another primer.

3) Off-target amplification caused by high-fidelity polymerase. Generally, as long as the DNA sequence with off-target binding of primer has mis-matched bases at the 3′ end, the amplification efficiency will be significantly reduced. However, in case of using a high-fidelity polymerase, the 3′-5′ exonuclease region of DNA polymerase will remove the mismatched bases, so that the DNA sequence with off-target binding of primers can also be amplified, finally forming a large number of off-target products.

### SUMMARY OF THE INVENTION

The present disclosure provides a method and a kit for amplifying a DNA target region.

The present disclosure provides a method for amplifying a DNA target region, including: linearly amplifying a fragmented DNA including the target region by a specific primer to provide a linear amplification product, where 3′ end of the specific primer is modified by a duality functional group, a phosphodiester bond of a part of nucleotide backbone at the 3′ end of the specific primer is modified by phosphorothioate, and the duality functional group is used to prevent the 3′ end of the specific primer from ligation with other oligonucleotides and can be removed by a specific enzyme to perform a linear amplification of the specific primer.

The present disclosure provides a method for constructing a library, including constructing the library by the linear amplification product as provided above.

The present disclosure provides a method for sequencing a DNA target region, including: sequencing the library product as provided above to provide a sequencing result of the target region.

The present disclosure provides a kit for amplifying a DNA target region, the kit being applicable to the method for amplifying a DNA target region as described above, or the method for constructing a library as described above, or the method for sequencing a DNA target region as described above.

### BRIEF DESCRIPTION OF THE DRAWINGS

FIG. 1 shows a schematic diagram of the number of reads obtained by library construction from the linear amplification with different primers in Embodiment 1 of the present disclosure.

FIG. 2 shows a schematic diagram of the proportion of reads obtained by library construction from the linear amplification with different primers in Embodiment 1 of the present disclosure.

FIG. 3 is a schematic diagram showing the proportion of specific library molecules obtained by linear amplification with different primers in Embodiment 1 of the present disclosure.

FIG. 4 shows a schematic diagram of the number of reads obtained by library construction from the linear amplification with different primers in Embodiment 2 of the present disclosure.

FIG. 5 shows a schematic diagram of the proportion of reads obtained by library construction from the linear amplification with different primers in Embodiment 2 of the present disclosure.

FIG. 6 is a schematic diagram showing the proportion of specific library molecules obtained by linear amplification with different primers in Embodiment 2 of the present disclosure.

### DETAILED DESCRIPTION

In order to make the inventive object, technical solutions and beneficial technical effects of the present disclosure clearer, hereinafter the present disclosure is further described in detail by reference to the embodiments. Persons skilled in the art can easily understand other advantages and other efficacies of the present disclosure from disclosure of the disclosure as claimed in the present specification.

By a great deal of practical researches, the inventor provides a novel method for amplifying a DNA target region. By introducing a phosphorothioate modification into specific primers, the non-specific amplification during the linear amplification is effectively reduced, the amplification product obtained by the linear amplification can be further used to construct a library, and the library data quality of the constructed library can meet the requirements. On that basis, the present disclosure is completed.

A first aspect of the present disclosure provides a method for amplifying a DNA target region, including: linearly amplifying a fragmented DNA including the target region by a specific primer to provide a linear amplification product, where 3′ end of the specific primer is modified by a duality functional group, a phosphodiester bond of a part of nucleotide backbone of the 3′ end of the specific primer is modified by phosphorothioate, and the duality functional group is used to prevent the 3′ end of the specific primer from ligation with other oligonucleotides and can be removed by a specific enzyme so that the linear amplification of the specific primer can be performed. In the present disclosure, the phosphorothioate modification of nucleotide in the specific primer means in particular that the phosphodiester bond in the nucleotide backbone is converted from P═O double bond to P═S double bond, which is specifically represented by a structural as shown in the following structural formula. As described above, by introducing a phosphorothioate modification into the specific primers for linear amplification, the proportion of the quality-controlled library molecules can be effectively increased, showing that the non-specific amplification can be significantly reduced during linear amplification.

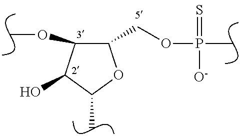

In the method for amplifying a DNA target region provided in the present disclosure, the linear amplification can generally be multiplexed linear amplification, and the amplification system can generally include multiple specific primers targeting different target regions, e.g., the number of the target regions as targeted is ≤2, 2 to 3, 3 to 4, 4 to 5, 5 to 6, 6 to 8, 8 to 10, 10 to 15, 15 to 20, or more. In general, the specific primer can include a sequence at least partially complementary to the target region of the fragmented DNA to achieve the specific amplification of the target region of the fragmented DNA. The skilled in the art can select a suitable target region of the fragmented DNA, and design a suitable complementary sequence. For example, the length of the sequence complementary to the target region can be ≤16 nt, 16 to 45 nt, 16 to 20 nt, 20 to 25 nt, 25 to 30 nt, 30 to 35 nt, 35 to 40 nt, or 40 to 45 nt in the specific primer. As another example, the specific primer further includes one or a combination of a first universal sequence (e.g., SP1 of the Illumina sequencing system), a first sample index (e.g., i5 of the Illumina sequencing system), and a first sequencing sequence (e.g., P5 of the Illumina sequencing system), and the like.

In the method for amplifying a DNA target region provided in the present disclosure, suitable methods for obtaining a fragmented DNA should be known to those skilled in the art. For example, the fragmented DNA containing a target region can generally originate from a genomic DNA. As another example, the fragmented DNA can be prepared from a genomic DNA via (random) disruption (e.g., by sonication or digestion). As another example, the fragmented DNA can be a free DNA, which can originate from body fluid, such as, blood and/or urine, etc. As another example, the fragmented DNA can have a structure of double-stranded DNA, single-stranded DNA and cDNA. For a linear amplification system, the fragmented DNA is generally required to have a suitable length, e.g., the fragmented DNA can be in length of 25 to 500 bp/nt, 25 to 30 bp/nt, 30 to 40 bp/nt, 40 to 50 bp/nt, 50 to 60 bp/nt, 60 to 80 bp/nt, 80 to 100 bp/nt, 100 to 150 bp/nt, 150 to 200 bp/nt, 200 to 300 bp/nt, 300 to 400 bp/nt, or 400 to 500 bp/nt.

In the method for amplifying a DNA target region provided in the present disclosure, the amplification system of the linear amplification can generally include the specific primer as described above, a DNA polymerase and dNTP. The linear amplification reaction of the fragmented DNA containing a target region by the specific primer can generally be performed in the presence of the DNA polymerase and/or dNTP. The DNA polymerase as used can generally have 3′-5′ exonuclease activity, so that the substituent group (e.g., the duality functional group) at the 3′ end of the primer bound to the template can be removed to activate the primer for conducting an extension, and the nucleotides with base mismatching can be removed during the DNA amplification to ensure the sequence accuracy of the amplified DNA. For example, the DNA polymerase as used can be Family B DNA polymerase. dNTPs (e.g., dATP, dGTP, dTTP, dCTP, etc.) are essential components in the linear amplification. At least part of the dNTPs can be dNTPs coupled with a labeling molecule which can be biotin or the like. By coupling with the labelling molecule, the amplification product can be purified.

In the method for amplifying a DNA target region provided in the present disclosure, the linear amplification process can generally include steps such as denaturation, annealing, and extension, and can further include steps such as pre-denaturation. In the annealing step of the linear amplification process, annealing generally requires a higher temperature. For example, the annealing temperature can be ≤60° C., 60-61° C., 61-63° C., 63-65° C., 65-67° C., 67-69° C., 69-71° C., 71-73° C., or 73-75° C. A high annealing temperature can ensure the specific binding of the primers. Since the primer sequence is present only at one end in the linear amplification, which has lower specificity than the PCR in which the primer sequences are present at both ends, it is required to increase the annealing temperature to achieve a suitable binding specificity of primers.

In the method for amplifying a DNA target region provided in the present disclosure, the specificity and/or uniformity of the multiplexed linear amplification is associated with the amount of the phosphorothioate modifications on the primer. When the amount of phosphorothioate modifications is too high or too low, the specificity of linear amplification will decrease, while when the amount of phosphorothioate-modification is relatively high, the linear amplification will have a relatively good uniformity. In general, the number of phosphorothioate-modified nucleotides can be 1 to 11, 1, 2, 3, 4, 5, 6, 7, 8, 9, 10 or 11, preferably 3 to 8. For the distribution positions of the phosphorothioate-modified nucleotides, the phosphorothioate-modified nucleotides can generally be contiguous (i.e., the positions of the phosphorothioate-modified nucleotides are contiguous on the primer) or dis-contiguous in the specific primer. Alternatively, at least part of the phosphorothioate-modified nucleotides can be located at the 3′ end of the specific primer, thereby inhibiting the interaction between the primers. The number of phosphorothioate-modified nucleotides located at the 3′ end of the specific primer can be 1 to 11, 1, 2, 3, 4, 5, 6, 7, 8, 9, 10, or 11, preferably 3 to 8.

In the method for amplifying a DNA target region provided in the present disclosure, the hydroxyl group attached to the 3-position C atom of the nucleotide at the 3′ end of the specific primer can be replaced by a duality functional group to prevent the 3′ end of the specific primer from ligation with other oligonucleotides and can be removed by a specific enzyme to allow the linear amplification reactions of specific primers. Persons skilled in the art can select a suitable duality functional group to achieve the modification of the 3′ end of the specific primer. For example, the modification group can replace the natural group (e.g., hydroxyl or the like) on the nucleotide at the 3′ end of the specific primer to prevent the 3′ end of the specific primer from ligation. After the primer is bound to the target region on a template via the complementary sequence to form a double-stranded structure, the duality functional group at the 3′ end of the primer can be removed by an enzyme to activate the primer, so that the target sequence can be effectively extended. For example, the duality functional group can be a C3 Spacer group, an Invert T group, a phosphate group, a biotin group, a C6 Spacer group, an NH₂—C6 group, or an SH—C6 group. As another example, the duality functional group can be a nucleotide complex group having a chemical structural formula of:

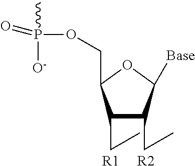

where, the Base can be any of adenine (base A), guanine (base G), cytosine (base C), thymine (base T) or uracil (base U);
R1 can be a hydroxyl group (—OH), C3 Spacer group, Invert T group, phosphate group (—PO₃), biotin group, C6 Spacer group, NH₂—C6 group or SH—C6 group;
R2 can be a hydrogen atom (—H), a fluorine atom (—F), a hydroxyl group (—OH) or a methoxy group (—OCH₃).

In a specific embodiment of the present disclosure, the nucleotide complex group can be any of DL1 to DL16, and the specific combination of the groups involved in DL1 to DL16 are shown in Table 1.

TABLE 1

| Name of Nucleotide | | | |
|---|---|---|---|
| Complex Group | Base | R1 | R2 |
| DL1 | base T | NH2-C6 group | hydrogen atom |
| DL2 | base T | NH2-C6 group | hydrogen atom |
| DL3 | base C | NH2-C6 group | hydrogen atom |
| DL4 | base G | NH2-C6 group | hydrogen atom |
| DL5 | base T | biotin group | hydrogen atom |
| DL6 | base T | C6 Spacer Group | hydrogen atom |
| DL7 | base T | Invert T group | hydrogen atom |
| DL8 | base T | Phosphate group | hydrogen atom |
| DL9 | base T | C3 Spacer Group | hydrogen atom |
| DL10 | base T | SH-C6 group | hydrogen atom |
| DL11 | base C | hydroxyl group | methoxy group |
| DL12 | base U | hydroxyl group | fluorine atom |
| DL13 | base U | hydroxyl group | hydroxyl group |
| DL14 | base A | hydroxyl group | methoxy group |
| DL15 | base G | hydroxyl group | methoxy group |
| DL16 | base U | hydroxyl group | methoxy group |

* The difference between DL1 and DL2 is that DL1 has no LNA modification, while DL2 has LNA modification.

In a specific embodiment of the present disclosure, when the duality functional group is a C3 Spacer group, a chemical structure as shown below can be formed:

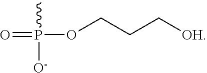

In a specific embodiment of the present disclosure, when the duality functional group is an Invert T group, a chemical structure as shown below can be formed:

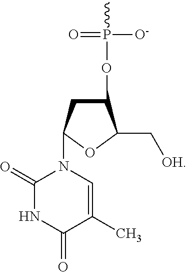 [SMILES: Cc1cn([C@H]2C[C@@H](OP(C)(=O)[O-])[C@@H](CO)O2)c(=O)[nH]c1=O]

In a specific embodiment of the present disclosure, when the duality functional group is a phosphate group, a chemical structure as shown below can be formed:

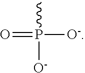

In a specific embodiment of the present disclosure, when the duality functional group is a biotin group, a chemical structure as shown below can be formed:

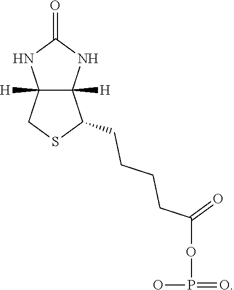

In a specific embodiment of the present disclosure, when the duality functional group is a C6 Spacer group, a chemical structure as shown below can be formed:

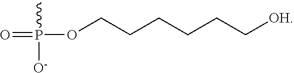

In a specific embodiment of the present disclosure, when the duality functional group is an NH2-C6 group, a chemical structure as shown below can be formed:

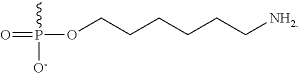

In a specific embodiment of the present disclosure, when the duality functional group is an SH—C6 group, a chemical structure as shown below can be formed:

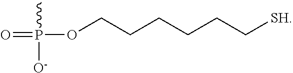

The method for amplifying a DNA target region can further include: purifying the linear amplification product. The skilled in the art can select a suitable method to purify the linear amplification product. For example, as described above, at least part of the dNTPs can be dNTPs coupled to a labelling molecule, the coupled labelling molecule can be biotin or the like, and the specific purification method can be affinity purification for labelling molecules of the dNTPs, etc.

In a specific embodiment of the present disclosure, the nucleotide sequence of the specific primer includes one or a combination of the sequences shown in SEQ ID NOs: 1-10.

A second aspect of the present disclosure provides a method for constructing a library, including: constructing a library using the linear amplification product provided in the first aspect of the present disclosure. Suitable methods for constructing a library from the linear amplification product as described above should be known to those skilled in the art. For example, it can include steps such as adapter ligation, pre-amplification, and library amplification.

The method for constructing a library provided in the present application may include: ligating the resultant linear amplification product to a single-chain adapter to obtain a ligation product, where the single-chain adapter includes a second sequencing sequence (e.g., P7 of the Illumina sequencing system) and/or a second sample index sequence (e.g., i7 of Illumina sequencing system) and/or a second universal sequence (e.g., SP2 of Illumina sequencing system) and/or a unique molecular index sequence (a random sequence for labelling each DNA molecule in the original sample). After obtaining the linear amplification product for the target region, a adapter can be further ligated to the linear amplification product to facilitate further sequencing.

In the method for constructing a library provided in the present disclosure, the skilled in the art can generally ligate a suitable single-chain adapter to the linear amplification product according to the subsequent sequencing method as required. For example, the single-chain adapter can include a second sequencing sequence and/or a second sample index sequence and/or a second universal sequence and/or a molecular index sequence. As another example, the single-stranded ligase for use in the adapter ligation can be a T4 RNA ligase or a thermostable RNA ligase (although RNAse is used, what is ligated is a DNA molecule). As another example, the nucleotide at the 5′ end of the single-chain adapter is modified and has a single-stranded structure at the reaction temperature of the adapter ligation. Specifically, a phosphate group or an adenosine group is attached to the 5-position C atom of the nucleotide at the 5′ end of the single-chain adapter. As another example, the hydroxyl group attached to the 3-position carbon atom of the nucleotides at the 3′ end of the single-chain adapter is replaced by a blocking group. Specially, the blocking group at the 3′ end of the single-chain adapter is selected from the group consisting of Invert T group, phosphate group, biotin group, C6 Spacer group, NH2-C6 group, SH—C6 group, and C3 Spacer group, and the chemical groups formed therefrom can refer to the chemical structures as provided above. As another example, the 5′-end region of the single-chain adapter is generally a partially double-stranded structure with cohesive ends.

In a specific embodiment of the present disclosure, the nucleotide sequence of the single-chain adapter includes one or more of the sequences shown in SEQ ID NOs: 11-12.

The method for constructing a library provided in the present disclosure can further include: pre-amplifying the ligation product to provide a pre-amplification product. The skilled in the art can generally select suitable conditions to pre-amplify the ligation product according to the subsequent sequencing method as required, and purify the pre-amplification product (e.g., by magnetic bead purification, etc.). For example, the reaction system of the pre-amplification can include pre-amplification primers, a DNA polymerase and dNTPs, where the DNA polymerase is preferably a B family DNA polymerase, and the forward primer of the pre-amplification primers includes a sequence complementary to the first sequencing sequence, and/or, the forward primer of the pre-amplification primers further includes a sequence complementary to the first universal sequence, and/or, the forward primer of the pre-amplification primers further includes a sequence complementary to the first sample index sequence, and the reverse primer of the pre-amplification primers includes a sequence complementary to the second sequencing sequence.

The method for constructing a library provided in the present disclosure can further include: expanding the pre-amplification product to provide a library amplification product

The skilled in the art can generally select suitable conditions to expand the pre-amplified product and purify the library amplification product (e.g., by magnetic bead purification, etc.) according to the subsequent sequencing method as required. For example, the reaction system of the library amplification system can include library amplification primers, a DNA polymerase and dNTPs, where the DNA polymerase is preferably a Family B DNA polymerase, and the forward primer of the library amplification primers includes a sequence complementary to the first sequencing sequence, and the reverse primer of the library amplification primers includes a sequence complementary to the second sequencing sequence.

A third aspect of the present disclosure provides a method for sequencing a DNA target region, including: sequencing a library amplification product by the library provided in the second aspect of the present disclosure to provide a sequencing result of the target region. The skilled in the art can generally select suitable conditions to sequence (e.g., using various second-generation sequencing technologies) the library amplification products according to the subsequent sequencing method as required. Preferably, the library molecule has a structure of:

| | | | | | | | | | |
|---|---|---|---|---|---|---|---|---|---|
| 5′ | 1^{st} | 1^{st} | 1^{st} | Sequence | Unique | 2^{nd} | 2^{nd} | 2^{nd} | 3′ |
| | sequencing | sample | universal | of Target | Molecule | universal | sample | sequencing | |
| | sequence | index | sequence | Region | index | sequence | index | sequence | |
| | | sequence | | | sequence | | sequence | | |

Of those, the first sequencing sequence can be P5 of the Illumina sequencing system, the first/second sample index sequence can be i5 or i7 of the Illumina sequencing system, the first/second universal sequence can be SP1 or SP2 of the Illumina sequencing system, and the second sequencing sequence can be P7 of the Illumina sequencing system.

A fourth aspect of the present disclosure provides a kit for use in a method for amplifying a DNA target region, which is suitable to the method for amplifying a DNA target region provided in the first aspect of the present disclosure, or the method for constructing a library provided in the second aspect of the present disclosure, or a method for sequencing a DNA target region provided in the third aspect of the present disclosure. The kit can include various specific primers as described above, and the phosphodiester bond of a part of the nucleotide backbone at the 3′ end of the specific primer can be modified by phosphorothioate. The kit can further include components essential to the reaction system of the linear amplification, such as, DNA polymerase and dNTPs.

In the method for amplifying a DNA target region provided in the present disclosure, the non-specific amplification during the linear amplification process is effectively reduced by introducing a phosphorothioate modification into the specific primer; a duality functional group modification is introduced into the specific primer to prevent the 3′ end of the specific primer from ligation with other oligonucleotides, which is efficiently removed by a DNA polymerase with 3′-5′ exonuclease activity after the specific primer binds to the template, to achieve efficient primer extension; and the amplification product obtained by the linear amplification can be further used to construct a library, and the data quality of the constructed library can meet the requirements, therefore, the present disclosure has a good industrialization prospect.

Hereinafter, the present disclosure will be further described by reference to embodiments, but the scope of the present disclosure will not be limited thereby.

Unless otherwise stated, the experimental methods, detection methods, and preparation methods disclosed in the present disclosure all adopt conventional technologies in the fields of molecular biology, biochemistry, chromatin structure and assay, analytical chemistry, cell culture, recombinant DNA technology and related fields. These techniques have been fully described in the existing literature. Specially, refer to Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999 et al.

### Embodiment 1

Comparison of Phosphorothioate-Primer and Non-Phosphorothioate-Primer with Respect to Library Construction Success Rate and Specificity of Multiplexed Linear Amplification

Multiplexed linear amplification was carried out with a phosphorothioate-primer and a non-phosphorothioate-primer, respectively. Normal human genomic DNA was interrupted by 260S sonication, and the fragmented DNA samples were subject to library construction. The library construction success rate and specificity of the phosphorothioate-primer and the non-phosphorothioate-primer were compared, demonstrating that the phosphorothioate-primer was suitable for high-specificity target library construction.

### Experimental Materials

### 1. Test Samples

The samples are fragmented DNA samples of normal human genomic DNA interrupted by 260 sec ultrasound.

The samples are quantified using Qubit, and the concentration of the sample was set as 20 ng/μL. The test is repeated 10 times.

### 2. The Primer Sequences are Shown in Table 2. The Suppliers are all Sangon Biotech (Shanghai) Co., Ltd.

TABLE 2

| | | Concentration | |
|---|---|---|---|
| No. | Primer Name | (nM) | Primer Sequence |
| 1 | ALK_f19-12N | 100 | TCGTCGGCAGCGTCAGATGTGTATAAGAGACAGC |
| | | | CTGATCAGCCAGGAGGATACACACG (SEQ ID NO: |
| | | | 1) |
| 2 | ALK_f19-4N | 50 | TCGTCGGCAGCGTCAGATGTGTATAAGAGACAGT |
| | | | GCCAAGCCACAGAGTTGGAGAAGAG (SEQ ID NO. |
| | | | 2) |
| 3 | ALK_f19-n4N | 100 | TCGTCGGCAGCGTCAGATGTGTATAAGAGACAGC |
| | | | AAGTATAACCCCACGTGAACGAG (SEQ ID NO. 3) |
| 4 | B26_i01-2N | 200 | TCGTCGGCAGCGTCAGATGTGTATAAGAGACAGT |
| | | | GGATATTGCAGCAGTCAGAGCCCTTAAC (SEQ ID |
| | | | NO. 4) |
| 5 | BRAF_i15-2N | 100 | TCGTCGGCAGCGTCAGATGTGTATAAGAGACAGG |
| | | | ATCCAGACAACTGTTCAAACTGATGGG (SEQ ID |
| | | | NO. 5) |
| 6 | EGFR_i21-2N | 100 | TCGTCGGCAGCGTCAGATGTGTATAAGAGACAGC |
| | | | CTGGCAGCCAGGAACGTACTGGTGAAAAC (SEQ ID |
| | | | NO. 6) |
| 7 | KRAS_i02-2N | 300 | TCGTCGGCAGCGTCAGATGTGTATAAGAGACAGG |
| | | | GCCTGCTGAAAATGACTGAATATAAACTTGTG (SEQ |
| | | | ID NO. 7) |
| 8 | MET_i14-1N | 50 | TCGTCGGCAGCGTCAGATGTGTATAAGAGACAGT |
| | | | GCCCGAAGTGTAAGCCCAACTACAG (SEQ ID NO. |
| | | | 8) |
| 9 | PIK3CA_i02- | 100 | TCGTCGGCAGCGTCAGATGTGTATAAGAGACAGT |
| | 1N | | CTCGATTGAGGATCTTTTCTTCACGGTTGC (SEQ ID |
| | | | NO. 9) |
| 10 | TP53_i07-2N | 100 | TCGTCGGCAGCGTCAGATGTGTATAAGAGACAGA |
| | | | GTCTTCCAGTGTGATGATGGTGAGGATG (SEQ ID |
| | | | NO: 10) |

*The primers and sequences used in panel 1 and panel 2 are exactly the same, except that the primer for panel 2 does not have phosphorothioate modification, while the primer for panel 1 has phosphorothioate modifications at the phosphodiester bonds of the last 3 nucleotides at the 3′ end. The sequence in bold represents the first universal sequence, and the sequence in non-bold represents the sequence complementary to the target region. All primers are modified with DL1 duality functional group.

### 3. The Single-Chain Adapters and Pre-Amplification Primers are Shown in Table 3 in Detail.

TABLE 3

| | | Primers For | |
|---|---|---|---|
| Single- | | Library | |
| Chain | Sequence of Single-Chain | Pre- | Primer Sequences for Library |
| Adapters | Adapters | Amplification | Pre-Amplification |
| UA4 | GNNTGNNTGNNTGNNCTGT | i3 | AATGATACGGCGACCACCGA |
| | CTCTTATACACATCTCCGAG | | GATCTACACAGGATAGGTCG |
| | CCCACGAGACGGAATCTCA | | TCGGCAGCGTCAGATG (SEQ |
| | TCTCGTATGCCGTCTTCTG | | ID NO. 13) |
| | CTTG (SEQ ID NO. 11) | | |
| UA5 | GNNTGNNTGNNTGNNCTGT | i4 | AATGATACGGCGACCACCGA |
| | CTCTTATACACATCTCCGAG | | GATCTACACTTATGCGATCGT |
| | CCCACGAGACTTCTGAATA | | CGGCAGCGTCAGATG (SEQ |
| | TCTCGTATGCCGTCTTCTG | | ID NO. 14) |
| | CTTG (SEQ ID NO. 12) | | |

*In all the single-chain adapters, a phosphate group is attached to the 5-position C atom of the 5′ end nucleotide, and the nucleotide at the 3′ end is modified with a C3 Spacer group. The experiments for panel 1 and panel 2 were repeated in a total of 10 times, respectively, where the 10 repeated experiments for panel 1 are represented by P1-01 to P1-10, and the 10 repeated experiments for panel 2 are represented by P2-01 to P2-10. Every two experimental libraries form a group, and the samples are mixed and then sequenced on the machine. The libraries in the same group were constructed using the paired single-chain adapter and pre-amplification primer as shown in Table 2, so that the two libraries in a single sequencing result can be distinguished from each other during the bio-information analysis. In the single-chain adapters, the sequences in bold represent the molecular index sequences, the sequences in underline represent the second universal sequences, and the sequences in non-bold/non-underline/non-italic represent the second sample index sequences, and the sequences in italic represent the second sequencing sequences; and in the primers for pre-amplification, the sequences in bold represent the first sequencing sequences, the sequences in non-bold/non-underline represent the first sample index sequences, and the sequences in underline represent the sequence complementary to the first universal sequence.

### 4. Other Primers and Probes

TABLE 4

| Names of | | |
|---|---|---|
| Primer and | | |
| Probes | Suppliers | Sequence |
| P5-AMP | Sangon Biotech | AATGATACGGCGACCACCGAGATCTACAC |
| | (Shanghai) Co., Ltd. | (SEQ ID NO: 15) |
| SP-EGFR21-1 | Sangon Biotech | TACTGGTGAAAACACCGCA (SEQ ID NO: 16) |
| | (Shanghai) Co., Ltd. | |
| F4-SP1 | Sangon Biotech | TCGTCGGCAGCGTCAGATG (SEQ ID NO: 17) |
| | (Shanghai) Co., Ltd. | |
| P7-AMP | BiOligo | CAAGCAGAAGACGGCATACGAGAT (SEQ ID |
| | Biotechnology | NO: 18) |
| | (Shanghai) Co., Ltd. | |
| MGB-iSP2-1 | BiOligo | TCTCGTGGGCTCGGAGA (SEQ ID NO: 19) |
| | Biotechnology | |
| | (Shanghai) Co., Ltd. | |
| R-EGFR21 | BiOligo | TTCCGCACCCAGCAGTTT (SEQ ID NO: 20) |
| | Biotechnology | |
| | (Shanghai) Co., Ltd. | |
| MGB-EGFR21 | BiOligo | TGTCAAGATCACAGATTTGGGC (SEQ ID NO: |
| | Biotechnology | 21) |
| | (Shanghai) Co., Ltd. | |

*P5-AMP is a primer for library amplification, which is complementary to the first sequencing sequence; SP-EGFR21-1, F4-SP1, P7-AMP and R-EGFR21 are primers for use in the qPCR system, where SP-EGFR21-1 is a primer complementary to the sequence of EGFR exon 21, F4-SP1 is a primer complementary to the first universal sequence, P7-AMP is a primer complementary to the second sequencing sequence, R-EGFR21 is a primer complementary to the sequence of EGFR exon 21; and MGB-iSP2-1 and MGB-EGFR21 are probes for use in the qPCR system, where MGB-iSP2-1 is a probe complementary to the second universal sequence, and MGB-EGFR21 is a probe complementary to complementary to the sequence of EGFR exon 21.

### 5. Other Reagents

TABLE 5

| Reagent Name | Suppliers/Formulations |
|---|---|
| APO-Enchanted DNA Polymerase I | Apogenomics Co., Ltd. |
| 5X Apo Buffer | Apogenomics Co., Ltd. |
| APO biotin-dNTP mix 1 | Apogenomics Co., Ltd. |
| Blocking Reagent | Apogenomics Co., Ltd. |
| Streptavidin Magnetic Beads | Apogenomics Co., Ltd. |
| Buffer A | Apogenomics Co., Ltd. |
| Buffer B | Apogenomics Co., Ltd. |
| Buffer C | Apogenomics Co., Ltd. |
| Buffer D | Apogenomics Co., Ltd. |
| ssDNA ligase | Apogenomics Co., Ltd. |
| 10X ligase buffer | Apogenomics Co., Ltd. |
| MnCl₂ | Apogenomics Co., Ltd. |
| 5X SLA Buffer | Apogenomics Co., Ltd. |
| SLA High Fidelity DNA Polymerase | Apogenomics Co., Ltd. |
| dNTP Mix (10 mM) | Apogenomics Co., Ltd. |
| NA-Beads | Apogenomics Co., Ltd. |
| Buffer E | Apogenomics Co., Ltd. |
| Buffer F | Apogenomics Co., Ltd. |
| TE buffer | Tris. Cl 10 mM, EDTA 1 mM, |
| | pH 8.0 |
| Realtime PCR Master Mix | TOYOBO |
| Qubit dsDNA HS Assay kit | Invitrogen |
| QC calibrator | Apogenomics Co., Ltd. |

* APO-Enchanted DNA polymerase I is a DNA polymerase with 3′-5′ exonuclease activity, 5X Apo Buffer is a buffer for the linear amplification system, and APO biotin-dNTP mix 1 is a mixed solution of dNTPs partially coupled with biotin, the Blocking Reagent is a reagent for blocking the streptavidin magnetic beads, the streptavidin magnetic beads are magnetic beads for purifying the linear amplification products, the Buffers A-D are respectively a binding buffer, a washing buffer 1, a washing buffer 2 and an elution buffer for purifying the linear amplification product, ssDNA ligase is a single-stranded ligase, the 10X ligase buffer is a buffer for the adapter ligation system, MnCl₂ is a component of the adapter ligation system, 5X SLA Buffer is a buffer for the pre-amplification or library amplification, the dNTP Mix is a mixed solution for pre-amplification or library amplification, the NA-Beads are magnetic beads for purifying the pre-amplification products or library amplification products, Buffer E and F are a binding buffer and an elution buffer for the pre-amplification products or library amplification products, respectively, Realtime PCR Master Mix is a mixed solution for qPCR, Qubit dsDNA HS Assay kit is a kit for quantification of samples, and the QC calibrator is a sample for quality control.

### 6. Experimental Equipments

TABLE 6

| Equipment Name | Manufacturers |
|---|---|
| Veriti 96 Well Thermal Cycler I | Thermo |
| Centrifuge | Eppendorf |
| Handheld Centrifuge | Kylin-Bell |
| Magnetic Grate with 12 Holes/96 | Thermo |
| Holes | |
| 7300 plus Real-time PCR system | Thermo |
| Water Purification Equipment | PALL |

### Experimental Steps

The qPCR detection system is configured according to the following system to quantify the amount of initial quality control molecules.

TABLE 7

| Components | Volume (μL) | Final concentration |
|---|---|---|
| ddH2O | 6.6 | / |
| 2X Realtime PCR Master Mix | 10 | 1X |
| R-EGFR21 (10 μM) | 0.6 | 300 nM |
| SP-EGFR21-1 (10 μM) | 0.6 | 300 nM |
| MGB-EGFR21 (10 μM) | 0.2 | 100 nM |
| Fragmented DNA | 2 | / |
| samples/QC calibrators | | |
| Total | 20 | |

### Testing Procedures:

TABLE 8

| Number of Cycles | Temperature (° C.) | Time | |
|---|---|---|---|
| 1 | 95 | 4 | min |
| 45 | 95 | 10 | seconds |
| | 60 | 30 | seconds |

### 1. Linear Amplification

### 1.1 Preparation of Linear Amplification System

TABLE 9

| | Volume |
|---|---|
| Components | (μL) |
| 5X Apo Buffer | 4 |
| APO biotin-dNTP mix 1 | 0.8 |
| APO-Enchanted DNA Polymerase I | 0.2 |
| Total Mix solution | 5 |
| Combination of Primers (see Table 2 for 10 | 1 |
| primer sequences) | |
| Fragmented DNA samples | 1.5 (30 ng) |
| water | 12.5 |
| Total | 20 |

1) First, a Mix solution was prepared from 5×Apo Buffer, APO biotin-dNTP mix 1 and APO-Enchanted DNA polymerase I in accordance with the above table.

2) Subsequently, the combination of primers and the fragmented DNA sample were added, and diluted with water to a reaction volume of 20 μL for each sample.

3) The linear amplification procedure was initiated.

### 1.2 Settings of the Linear Amplification Conditions

TABLE 10

| Number of | | Temperature | |
|---|---|---|---|
| Cycles | Step | (° C.) | Time (s) |
| 1 | denaturation | 98 | 100 |
| 50 | denaturation | 95 | 10 |
| | annealing | 67 | 15 |
| | extension | 70 | 60 |
| 1 | temporary | 4 | / |
| | storage | | |

### 2. Purification of Linear Amplification Products

2.1 Preparation of Streptavidin Magnetic Beads (for Each Sample, 20 μL of Magnetic Beads were Added.)

1) The streptavidin magnetic beads were taken out of the 4° C. refrigerator and mixed with vortex for 30 seconds.

2) 1 mL of Buffer B was added and mixed by pipetting.

3) The mixture was placed on a 1.5 mL magnetic grate, allowed to stand for 2 min, and the supernatant was discarded.

4) 1 mL of Buffer B was added to wash the magnetic beads once.

5) A suitable volume of Buffer A was added for resuspension. 0.25 μL of Blocking Reagent was added to each sample, mixed well, and distributed into 8-tube strip.

6) The magnetic beads were blocked at room temperature at 500-600 rpm for 10 min.

### 2.2 Purification of Streptavidin Magnetic Beads

1) 20 μL of blocked magnetic beads were added to the 8-tube strip containing linear amplification products.

2) The 8-tube strip was shaken at 2000 rpm for 10-15 seconds until the magnetic beads were completely dispersed.

3) The mixture stood at room temperature at 500-600 rpm for 15 min, then was placed on a 96-well magnetic grate, and stood for additional 2 min. The supernatant was discarded.

4) 150 μL of Buffer B was added for washing the magnetic beads 3 times.

5) 150 μL of Buffer C was added for washing the magnetic beads twice.

6) 20 μL of Buffer D was added and shaken at 3500 rpm for 2-5 min until the magnetic beads were completely dispersed.

7) The target product was eluted at 80° C. for 4 min.

8) The eluate was heated, and then placed on the magnetic grate for 2-5 min. The supernatant was transferred to a new 8-tube strip for subsequent experiment.

### 3. Adapter Ligation

### 3.1 Preparation of Adapter Ligation System

TABLE 11

| Components | Volume (μL) |
|---|---|
| 10X ligase buffer | 2.5 |
| MnCl₂ | 0.63 |
| ssDNA ligase | 0.63 |
| Total mixture | 3.75 |
| Single-Chain Adapters | 1.25 |
| Purified linear amplification product | 20 |
| Total | 25 |

The single-chain adapters for the adapter ligation system (with different sample index sequence per sample) were added separately. According to the number of samples, the mixed solutions for the respective reaction system in addition to the single-chain adapter were prepared, and the single-chain adapters containing the respective sample index sequences (i.e., UA4 or UA5) were added to the respective reaction well according to the information sheet of library construction (Table 21).

### 3.2 Adapter Ligation Procedure

TABLE 12

| Number of Cycles | Temperature (° C.) | Time (minutes) |
|---|---|---|
| 1 (hot cover 105° C.) | 60 | 60 |
| 1 (hot cover 105° C.) | 90 | 3 |
| 1 | 8 | / |

### 3.3 Detection of Ligation Products

After ligation, 2 μL of the ligation product was taken, and 18 μL of TE buffer was added for 10× dilution. The dilution was used for detecting the efficiency of library construction. The efficiency of library construction was quantified by qPCR.

The qPCR detection system is configured according to the following system to quantify the number of quality control library molecules.

TABLE 13

| Components | Volume (μL) | Final Concentration |
|---|---|---|
| ddH2O | 6.6 | / |
| 2X Realtime PCR Master Mix | 10 | 1× |
| P7-AMP(10 μM) | 0.6 | 300 nM |
| SP-EGFR21-1 (10 μM) | 0.6 | 300 nM |
| MGB-iSP2-1(10 μM) | 0.2 | 100 nM |
| Ligation products/QC calibrators | 2 | / |
| Total | 20 | |

The detection procedure is the same as that in Table 8.

### 4. Pre-Amplification

### 4.1 Preparation of the Reaction System of Pre-Amplification:

TABLE 14

| Components | Volume (μL) | Final concentration | |
|---|---|---|---|
| ddH2O | 12.75 | / | |
| 5X SLA Buffer | 10 | 1× | |
| dNTP Mix | 1 | 200 | μM |
| Forward primers for the pre- | 1 | 100 | nM |
| amplification system | | | |
| P7-AMP | 1 | 200 | nM |
| SLA High Fidelity DNA Polymerase | 0.25 | 0.011 | U/μL |

| | |
|---|---|
| Total | 26 |
| The mixed solution was placed at | 26 μL/well |
| 4° C. | |
| Ligation product | 24 |
| Total | 50 |

4.1.1 The forward primers for the pre-amplification system (with different sample index sequence per sample) were added separately. According to the number of samples, the mixed solutions for the respective reaction system in addition to the forward primer were prepared in a 1.5 mL centrifuge tube, and distributed to an 8-tube strip. The forward primers containing the respective sample index sequences (i.e., i3 or i4) were added to the respective reaction well according to the information sheet of library construction (Table 21).

4.1.2 24 μL of ligation product was added to the respective reaction well. Then, the mixture was placed into the PCR instrument for reaction.

### 4.2 Pre-Amplification Procedure

TABLE 15

| Number of | | Temperature | | |
|---|---|---|---|---|
| Cycles | Step | (° C.) | Time | |
| 1 | denaturation | 95 | 3 | min |
| 5 | denaturation | 95 | 10 | seconds |
| | annealing | 70 | 10 | seconds |
| | extension | 72 | 30 | seconds |
| 1 | extension | 72 | 120 | seconds |

| | | |
|---|---|---|
| temporary | 8 | / |
| storage | | |

4.3 Purification of Pre-Amplification Products with NA-Beads

1) To the pre-amplification product was added an equal volume (50 μL) of Buffer E.

2) 70 μL of NA-Beads were added. The magnetic beads were mixed well by vortex shaking, and stood at 25° C. for 5 min.

3) The magnetic beads were adsorbed with a magnetic grate for 5 min until the solution was clear. The supernatant was carefully sucked and discarded.

4) 200 μL of 80% ethanol was added for washing twice, and finally, the supernatant was carefully removed to avoid sucking the magnetic beads.

5) After standing at room temperature for 3 min, 20 μL of Buffer F was added to fully suspend the magnetic beads. The mixture was placed at room temperature for 5 min to elute the DNA. The magnetic beads were adsorbed with a magnet, and the supernatant DNA solution was transferred to a new 8-tube strip. The The product was directly used for subsequent experiments.

### 5. Library Amplification

### 5.1 Preparation of the Reaction System of Library Amplification:

5.1.1 The respective mixed reaction solutions were prepared according to the table below. The mixed solution was distributed into an 8-tube strip.

TABLE 16

| | | Volume | | |
|---|---|---|---|---|
| Components | Concentration | (μL) | Final concentration | |
| ddH2O | | 2 | / | |
| 5X SLA Buffer | 5X | 6 | 1× | |
| dNTP Mix | (10 mM each) | 0.6 | 200 | μM |
| P5-AMP | 10 μM | 0.6 | 200 | nM |
| P7-AMP | 10 μM | 0.6 | 200 | nM |
| SLA High Fidelity | (2 U/μL) | 0.15 | 0.011 | U/μL |

| | |
|---|---|
| DNA Polymerase | |
| Total | 10 |
| The mixed solution | 10 μL/well |
| was placed at 4° C. | |
| Purified pre- | 20 |
| amplification product | |
| Total | 30 |

5.1.2 The respective purified pre-amplification product was added into the library amplification tube. The mixture was rapidly centrifuged for 10 seconds and placed in a PCR instrument for reaction.

### 5.2 Reaction Procedure for Library Amplification:

TABLE 17

| Number of Cycles | Step | Temperature (° C.) | Time | |
|---|---|---|---|---|
| 1 | denaturation | 95 | 3 | min |
| 12 | denaturation | 95 | 10 | seconds |
| | annealing | 72 | 10 | seconds |
| | extension | 72 | 30 | seconds |
| 1 | extension | 72 | 120 | seconds |

| | | |
|---|---|---|
| temporary | 8 | / |
| storage | | |

5.3 Purification of NA-Beads after Library Amplification

27 μL of 0.9×NA-Beads were added and mixed well by vortex shaking. The mixture stood at room temperature for 5 min.

1) The magnetic beads were adsorbed with a magnetic grate for 5 min until the solution is clear. The supernatant was carefully sucked and discarded.

2) 200 μL of 80% ethanol was added for washing twice, and finally, the supernatant was carefully removed to avoid sucking the magnetic beads.

3) After standing at room temperature for 3 min, 20 μL of Buffer F was added to fully suspend the magnetic beads. The mixture was placed at room temperature for 5 min to elute the DNA. The magnetic beads were adsorbed with a magnet, and the supernatant DNA solution was transferred to a new 8-tube strip. The product was directly used for subsequent experiments.

4) 1 μL of the purified library amplification product was added into a low TE buffer (Tris·Cl 10 mM, EDTA 0.1 mM, pH 8.0). The purified library amplification product was 10,000× diluted, and quantitatively detected by qPCR for quantification of the library amplification product.

The qPCR detection system is configured according to the following system to quantify the number of total library molecules.

TABLE 18

| | Volume | Final |
|---|---|---|
| Components | (μL) | concentration |
| ddH2O | 6.6 | / |
| 2X Realtime PCR Master Mix | 10 | 1× |
| F4-SP1(10 μM) | 0.6 | 300 nM |
| P7-AMP (10 μM) | 0.6 | 300 nM |
| MGB-iSP2-1(10 μM) | 0.2 | 100 nM |
| Purified library amplification | 2 | / |
| product/QC calibrator | | |
| Total | 20 | |

The detection procedure is the same as that in Table 8.

### 6. Library Sequencing

Illumina's NovaSeq6000 platform was used to perform 150 bp paired-end sequencing on the library. The number of reads, read proportion and specificity proportion were calculated through bioinformatics analysis.

### Experimental Results

1. The quality control results of the library after linear amplification of different primer combinations (the initial number of quality control molecules is the number of molecules detected by the qPCR system in Table 7, that is, the initial amount of DNA input; the number of quality control library molecules is based on the number of molecules detected by the qPCR system in Table 13; the number of total library molecules is the number of molecules detected by the qPCR system in Table 18; the proportion of quality control library molecules=the number of quality control library molecules/the number of total library molecules×100%):

TABLE 19

| | | Number of | Number of | | Proportion of |
|---|---|---|---|---|---|
| | | initial quality | quality control | Number of | quality control |
| Sample | Primer | control | Library | Total Library | library |
| number | combination | molecules | Molecules | Molecules | molecules |
| P1-01 | panel 1 | 10,000 | 7086 | 1.30E+05 | 5.47% |
| P1-02 | panel 1 | 10,000 | 9287 | 1.43E+05 | 6.50% |
| P1-03 | panel 1 | 10,000 | 9273 | 1.86E+05 | 4.99% |
| P1-04 | panel 1 | 10,000 | 9053 | 2.09E+05 | 4.34% |
| P1-05 | panel 1 | 10,000 | 11269 | 2.21E+05 | 5.10% |
| P1-06 | panel 1 | 10,000 | 7920 | 1.42E+05 | 5.58% |
| P1-07 | panel 1 | 10,000 | 9792 | 2.09E+05 | 4.69% |
| P1-08 | panel 1 | 10,000 | 9684 | 1.79E+05 | 5.41% |
| P1-09 | panel 1 | 10,000 | 7844 | 1.56E+05 | 5.02% |
| P1-10 | panel 1 | 10,000 | 10858 | 1.88E+05 | 5.78% |
| P2-01 | panel 2 | 10,000 | 2813 | 2.80E+05 | 1.00% |
| P2-02 | panel 2 | 10,000 | 2844 | 1.22E+06 | 0.23% |
| P2-03 | panel 2 | 10,000 | 2083 | 1.29E+05 | 1.62% |
| P2-04 | panel 2 | 10,000 | 3585 | 2.61E+06 | 0.14% |
| P2-05 | panel 2 | 10,000 | 3702 | 9.57E+06 | 0.04% |
| P2-06 | panel 2 | 10,000 | 3361 | 9.33E+06 | 0.04% |
| P2-07 | panel 2 | 10,000 | 4463 | 2.45E+06 | 0.18% |
| P2-08 | panel 2 | 10,000 | 3658 | 1.22E+06 | 0.30% |
| P2-09 | panel 2 | 10,000 | 4193 | 2.52E+06 | 0.17% |
| P2-10 | panel 2 | 10,000 | 2422 | 2.89E+06 | 0.08% |

It can be seen from the data in the above table that by using the primer combination with phosphorothioate modification (panel 1), more quality control library molecules can be obtained. At the same time, the proportion of quality control library molecules is high, which meets the expectation. In the primer combination without phosphorothioate modification (panel 2), the proportion of quality control library molecules was significantly lower, while the total number of library molecules was significantly higher, indicating that there were more non-specific amplifications during library construction.

2. Quantification of the total library molecules after library amplification (the number of library molecules after ligation=the number of total library molecules; the number of total library molecules after library amplification is the number of molecules after library amplification detected by the qPCR system in Table 18; the library amplification multiple=the number of total library molecules after library amplification/the number of total library molecules×100%; the copy number of sample/μL=the number of total library molecules after library amplification/20; the loading copy number is theoretically fed DNA amount after library amplification; and the loading volume μL=the loading copy number/the copy number of sample/μL):

TABLE 20

| | Number | Number of | | | | |
|---|---|---|---|---|---|---|
| | of library | total library | | | | |
| | molecules | molecules | Library | Copy | Loading | Loading |
| Sample | after | after library | Amplification | Number of | Copy | Volume |
| No. | ligation | amplification | Multiple | Sample/μL | Number | μL |
| P1-01 | 1.30E+05 | 1.72E+09 | 1.32E+04 | 8.58E+07 | 1.00E+09 | 11.65 |
| P1-02 | 1.43E+05 | 2.06E+09 | 1.44E+04 | 1.03E+08 | 1.00E+09 | 9.71 |
| P1-03 | 1.86E+05 | 2.09E+09 | 1.12E+04 | 1.04E+08 | 1.00E+09 | 9.58 |
| P1-04 | 2.09E+05 | 2.68E+09 | 1.28E+04 | 1.34E+08 | 1.00E+09 | 7.46 |
| P1-05 | 2.21E+05 | 3.13E+09 | 1.42E+04 | 1.57E+08 | 1.00E+09 | 6.38 |
| P1-06 | 1.42E+05 | 1.58E+09 | 1.11E+04 | 7.90E+07 | 1.00E+09 | 12.66 |
| P1-07 | 2.09E+05 | 2.28E+09 | 1.09E+04 | 1.14E+08 | 1.00E+09 | 8.78 |
| P1-08 | 1.79E+05 | 2.09E+09 | 1.17E+04 | 1.04E+08 | 1.00E+09 | 9.57 |
| P1-09 | 1.56E+05 | 2.14E+09 | 1.37E+04 | 1.07E+08 | 1.00E+09 | 9.33 |
| P1-10 | 1.88E+05 | 2.44E+09 | 1.30E+04 | 1.22E+08 | 1.00E+09 | 8.21 |
| P2-01 | 2.80E+05 | 1.63E+09 | 5.83E+03 | 8.16E+07 | 1.00E+09 | 12.25 |
| P2-02 | 1.22E+06 | 1.11E+10 | 9.04E+03 | 5.53E+08 | 1.00E+09 | 1.81 |
| P2-03 | 1.29E+05 | 2.93E+09 | 2.28E+04 | 1.47E+08 | 1.00E+09 | 6.82 |
| P2-04 | 2.61E+06 | 2.48E+10 | 9.51E+03 | 1.24E+09 | 1.00E+09 | 0.81 |
| P2-05 | 9.57E+06 | 5.76E+10 | 6.02E+03 | 2.88E+09 | 1.00E+09 | 0.35 |
| P2-06 | 9.33E+06 | 6.97E+10 | 7.47E+03 | 3.49E+09 | 1.00E+09 | 0.29 |
| P2-07 | 2.45E+06 | 1.25E+10 | 5.12E+03 | 6.27E+08 | 1.00E+09 | 1.60 |
| P2-08 | 1.22E+06 | 8.11E+09 | 6.63E+03 | 4.05E+08 | 1.00E+09 | 2.47 |
| P2-09 | 2.52E+06 | 1.93E+10 | 7.69E+03 | 9.67E+08 | 1.00E+09 | 1.03 |
| P2-10 | 2.89E+06 | 2.20E+10 | 7.60E+03 | 1.10E+09 | 1.00E+09 | 0.91 |

It can be seen from the data in the above table that the library amplification efficiency of all the libraries meets the expectations.

3. Raw Data Quality:

TABLE 21

| Sample | Q30 | Single-Chain | Primers For Library Pre- |
|---|---|---|---|
| No. | (%) | Adapters | Amplification |
| P1-01 | 84.24 | UA4 | i3 |
| P1-02 | 79.83 | UA5 | i4 |
| P1-03 | 84.17 | UA4 | i3 |
| P1-04 | 85.59 | UA5 | i4 |
| P1-05 | 83.39 | UA4 | i3 |
| P1-06 | 84.04 | UA5 | i4 |
| P1-07 | 85.08 | UA4 | i3 |
| P1-08 | 89.13 | UA5 | i4 |
| P1-09 | 84.67 | UA4 | i3 |
| P1-10 | 83.94 | UA5 | i4 |
| P2-01 | 84.78 | UA4 | i3 |
| P2-02 | 85.52 | UA5 | i4 |
| P2-03 | 84.15 | UA4 | i3 |
| P2-04 | 87.84 | UA5 | i4 |
| P2-05 | 85.05 | UA4 | i3 |
| P2-06 | 82.75 | UA5 | i4 |
| P2-07 | 87.2 | UA4 | i3 |
| P2-08 | 84.99 | UA5 | i4 |
| P2-09 | 84.4 | UA4 | i3 |
| P2-10 | 84.24 | UA5 | i4 |

It can be seen from the data in the above table that the sequenced data quality of all the libraries meets the requirements.

4. Reads and proportions obtained by library construction from the linear amplification with different primers

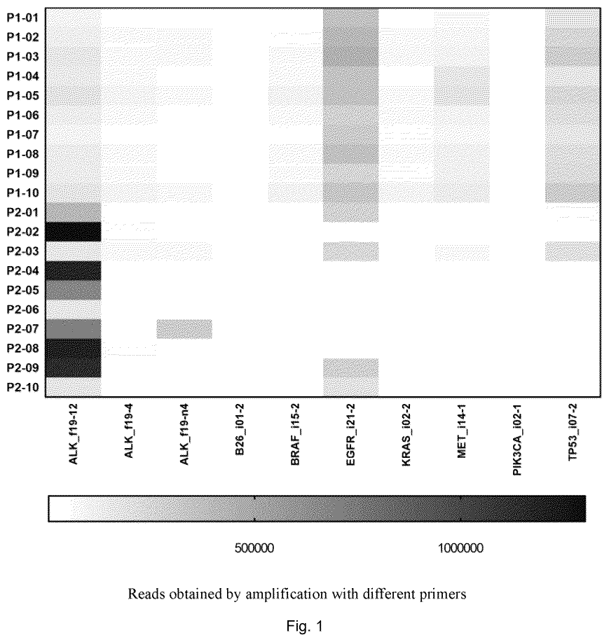

4.1. The number of reads obtained by library construction from the linear amplification with different primers. The results are shown in FIG. 1 and Table 22.

TABLE 22

| Sample | | ALK_{—} | ALK_{—} | ALK | B26_{—} | BRAF_{—} | EGFR_{—} | KRAS_{—} | MET_{—} | PIK3CA_{—} | TP53_{—} |
|---|---|---|---|---|---|---|---|---|---|---|---|
| No. | Total | f19-12 | f19-4 | f19-n4 | i01-2 | i15-2 | i21-2 | i02-2 | i14-1 | i02-1 | i07-2 |
| P1-01 | 830674 | 92178 | 36870 | 34646 | 6952 | 27475 | 353002 | 35728 | 54582 | 11293 | 177948 |
| P1-02 | 1099755 | 119693 | 55873 | 58120 | 11726 | 47099 | 402042 | 61492 | 78804 | 18932 | 245974 |
| P1-03 | 1273051 | 150469 | 61243 | 61766 | 13385 | 54971 | 442193 | 76168 | 96517 | 22389 | 293950 |
| P1-04 | 1014897 | 137471 | 55033 | 39264 | 14612 | 60705 | 345219 | 35082 | 149926 | 15933 | 161652 |
| P1-05 | 1277871 | 177262 | 84550 | 56264 | 16689 | 94775 | 356560 | 63896 | 174045 | 24118 | 229712 |
| P1-06 | 932951 | 123049 | 56535 | 44447 | 11099 | 56179 | 268757 | 55521 | 92846 | 16848 | 207670 |
| P1-07 | 827819 | 90201 | 45521 | 36625 | 9008 | 41857 | 303315 | 49201 | 73091 | 13236 | 165764 |
| P1-08 | 1052426 | 131381 | 61601 | 45185 | 12383 | 59129 | 359854 | 53631 | 103118 | 17074 | 209070 |
| P1-09 | 940226 | 132143 | 54839 | 44554 | 11888 | 52272 | 259638 | 50351 | 103982 | 15332 | 215227 |
| P1-10 | 1324740 | 170186 | 89994 | 64258 | 23059 | 92318 | 336761 | 88994 | 132713 | 26222 | 300235 |
| P2-01 | 846064 | 404398 | 42442 | 35428 | 3726 | 10805 | 264270 | 14896 | 17581 | 4073 | 48445 |
| P2-02 | 1415503 | 1301444 | 47337 | 14851 | 1887 | 4103 | 13356 | 4443 | 5456 | 1891 | 20735 |
| P2-03 | 857245 | 149678 | 65762 | 58756 | 13358 | 35520 | 232945 | 34540 | 62969 | 12281 | 191436 |
| P2-04 | 1234306 | 1186359 | 7090 | 14818 | 618 | 1827 | 9717 | 2025 | 2176 | 736 | 8940 |
| P2-05 | 692197 | 665479 | 2240 | 4763 | 445 | 1149 | 7338 | 1686 | 2075 | 431 | 6591 |
| P2-06 | 201106 | 154754 | 11560 | 5306 | 917 | 2239 | 7380 | 2409 | 2941 | 844 | 12756 |
| P2-07 | 1054312 | 704251 | 6624 | 300597 | 1465 | 3105 | 11362 | 3767 | 4532 | 1228 | 17381 |
| P2-08 | 1390635 | 1209016 | 46824 | 30529 | 3989 | 9732 | 23046 | 9543 | 12161 | 3592 | 42203 |
| P2-09 | 1468005 | 1146189 | 10537 | 10403 | 1187 | 2484 | 275940 | 3132 | 3737 | 998 | 13398 |
| P2-10 | 378775 | 170080 | 3078 | 5189 | 978 | 2310 | 180788 | 2853 | 3329 | 741 | 9429 |

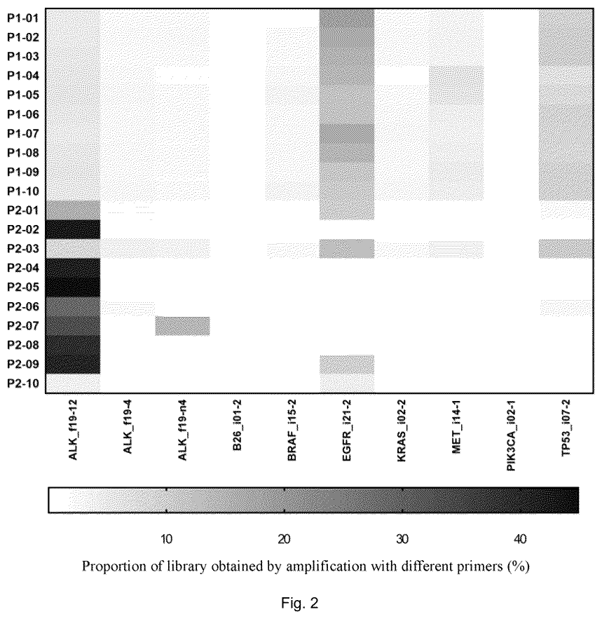

4.2. The results of the proportion of reads obtained by library construction from the linear amplification with different primers are shown in FIG. 2 and Table 23. The proportion of reads is calculated by dividing the number of reads for each primer by the number of total reads×100%.

TABLE 23

| Sample | ALK_{—} | ALK_{—} | ALK_{—} | B26_{—} | BRAF_{—} | EGFR_{—} | KRAS_{—} | MET_{—} | PIK3CA_{—} | TP53_{—} |
|---|---|---|---|---|---|---|---|---|---|---|
| No. | f19-12 | f19-4 | f19-n4 | i01-2 | i15-2 | i21-2 | i02-2 | i14-1 | i02-1 | i07-2 |
| P1-01 | 11.10% | 4.44% | 4.17% | 0.84% | 3.31% | 42.50% | 4.30% | 6.57% | 1.36% | 21.42% |
| P1-02 | 10.88% | 5.08% | 5.28% | 1.07% | 4.28% | 36.56% | 5.59% | 7.17% | 1.72% | 22.37% |
| P1-03 | 11.82% | 4.81% | 4.85% | 1.05% | 4.32% | 34.73% | 5.98% | 7.58% | 1.76% | 23.09% |
| P1-04 | 13.55% | 5.42% | 3.87% | 1.44% | 5.98% | 34.02% | 3.46% | 14.77% | 1.57% | 15.93% |
| P1-05 | 13.87% | 6.62% | 4.40% | 1.31% | 7.42% | 27.90% | 5.00% | 13.62% | 1.89% | 17.98% |
| P1-06 | 13.19% | 6.06% | 4.76% | 1.19% | 6.02% | 28.81% | 5.95% | 9.95% | 1.81% | 22.26% |
| P1-07 | 10.90% | 5.50% | 4.42% | 1.09% | 5.06% | 36.64% | 5.94% | 8.83% | 1.60% | 20.02% |
| P1-08 | 12.48% | 5.85% | 4.29% | 1.18% | 5.62% | 34.19% | 5.10% | 9.80% | 1.62% | 19.87% |
| P1-09 | 14.05% | 5.83% | 4.74% | 1.26% | 5.56% | 27.61% | 5.36% | 11.06% | 1.63% | 22.89% |
| P1-10 | 12.85% | 6.79% | 4.85% | 1.74% | 6.97% | 25.42% | 6.72% | 10.02% | 1.98% | 22.66% |
| P2-01 | 47.80% | 5.02% | 4.19% | 0.44% | 1.28% | 31.24% | 1.76% | 2.08% | 0.48% | 5.73% |
| P2-02 | 91.94% | 3.34% | 1.05% | 0.13% | 0.29% | 0.94% | 0.31% | 0.39% | 0.13% | 1.46% |
| P2-03 | 17.46% | 7.67% | 6.85% | 1.56% | 4.14% | 27.17% | 4.03% | 7.35% | 1.43% | 22.33% |
| P2-04 | 96.12% | 0.57% | 1.20% | 0.05% | 0.15% | 0.79% | 0.16% | 0.18% | 0.06% | 0.72% |
| P2-05 | 96.14% | 0.32% | 0.69% | 0.06% | 0.17% | 1.06% | 0.24% | 0.30% | 0.06% | 0.95% |
| P2-06 | 76.95% | 5.75% | 2.64% | 0.46% | 1.11% | 3.67% | 1.20% | 1.46% | 0.42% | 6.34% |
| P2-07 | 66.80% | 0.63% | 28.51% | 0.14% | 0.29% | 1.08% | 0.36% | 0.43% | 0.12% | 1.65% |
| P2-08 | 86.94% | 3.37% | 2.20% | 0.29% | 0.70% | 1.66% | 0.69% | 0.87% | 0.26% | 3.03% |
| P2-09 | 78.08% | 0.72% | 0.71% | 0.08% | 0.17% | 18.80% | 0.21% | 0.25% | 0.07% | 0.91% |
| P2-10 | 44.90% | 0.81% | 1.37% | 0.26% | 0.61% | 47.73% | 0.75% | 0.88% | 0.20% | 2.49% |

It can be seen from FIGS. 1 to 2 and Tables 22 to 23 that the amplification products obtained by the combination of multiple primers with no phosphorothioate modification has poor amplification uniformity, and most of them are the amplification product of individual primers. In 70% of the detected samples, the phenomenon that the libraries from a single primer primary accounted for more than 50%, and these samples showed remarkable non-specific PCR products. However, the amplification products obtained by the combination of multiple primers with phosphorothioate modification has superior amplification uniformity to the combination with non phosphorothioate modification, and no remarkable PCR product formed from a primer occurs.

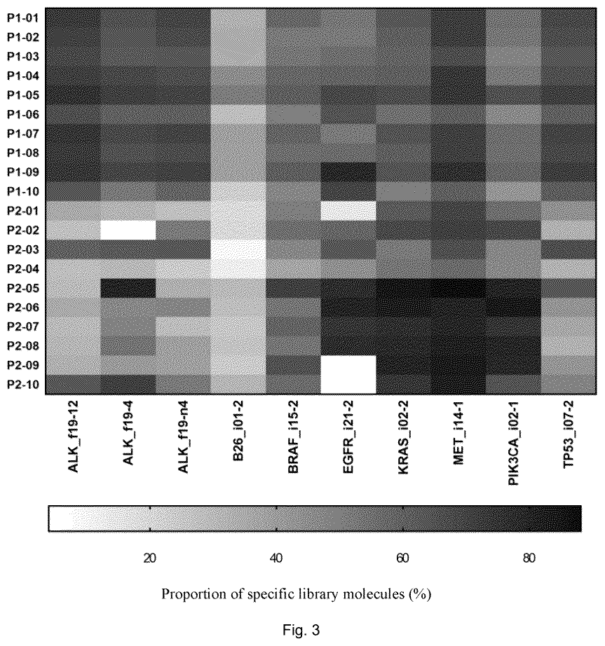

5. The results of proportion of specificity of library molecules obtained by the linear amplification with different primers are shown in FIG. 3 and Table 24. The proportion of specificity is calculated by dividing the number of ontarget reads for each primer by the number of reads for each primer (that is, the number of reads obtained by library construction from the linear amplification with the respective primers in Table 22)×100%.

TABLE 24

| Sample | ALK_{—} | ALK_{—} | ALK_{—} | B26_{—} | BRAF_{—} | EGFR_{—} | KRAS_{—} | MET_{—} | PIK3CA_{—} | TP53_{—} |
|---|---|---|---|---|---|---|---|---|---|---|
| No. | f19-12 | f19-4 | f19-n4 | i01-2 | i15-2 | i21-2 | i02-2 | i14-1 | i02-1 | i07-2 |
| P1-01 | 72.66% | 65.23% | 69.31% | 32.48% | 57.96% | 52.51% | 62.86% | 71.67% | 54.06% | 68.75% |
| P1-02 | 70.60% | 63.34% | 67.63% | 33.66% | 51.88% | 52.12% | 60.11% | 72.06% | 52.56% | 66.57% |
| P1-03 | 68.34% | 62.20% | 62.84% | 33.33% | 52.50% | 55.86% | 58.84% | 66.39% | 48.54% | 63.15% |
| P1-04 | 71.33% | 68.67% | 66.80% | 45.09% | 58.31% | 59.22% | 60.17% | 73.76% | 52.07% | 65.65% |
| P1-05 | 75.83% | 70.93% | 69.78% | 50.72% | 61.24% | 68.56% | 66.83% | 74.18% | 65.01% | 70.87% |
| P1-06 | 65.82% | 59.92% | 60.27% | 28.43% | 49.63% | 64.41% | 54.87% | 60.87% | 45.94% | 60.59% |
| P1-07 | 74.66% | 67.68% | 69.76% | 38.67% | 58.88% | 51.71% | 64.63% | 71.24% | 55.78% | 69.84% |
| P1-08 | 72.83% | 65.08% | 67.07% | 38.44% | 56.05% | 56.25% | 60.91% | 70.76% | 55.39% | 68.16% |
| P1-09 | 74.13% | 69.22% | 70.40% | 38.55% | 61.17% | 79.12% | 64.52% | 75.95% | 57.99% | 71.84% |
| P1-10 | 63.97% | 52.36% | 58.88% | 22.89% | 47.08% | 69.09% | 49.34% | 60.50% | 42.07% | 61.47% |
| P2-01 | 35.53% | 31.97% | 27.89% | 18.38% | 49.88% | 13.84% | 61.87% | 69.26% | 56.00% | 43.73% |
| P2-02 | 27.94% | 5.41% | 51.28% | 19.02% | 55.76% | 58.44% | 67.81% | 70.84% | 68.22% | 32.57% |
| P2-03 | 59.74% | 62.08% | 62.40% | 8.59% | 46.96% | 63.34% | 51.78% | 65.50% | 46.92% | 66.00% |
| P2-04 | 28.28% | 30.24% | 23.72% | 12.14% | 36.51% | 44.01% | 52.99% | 56.43% | 46.47% | 32.04% |
| P2-05 | 29.07% | 79.87% | 33.45% | 29.44% | 71.02% | 77.77% | 84.88% | 88.10% | 80.28% | 63.40% |
| P2-06 | 35.02% | 46.49% | 47.64% | 28.35% | 52.93% | 80.84% | 82.86% | 80.14% | 84.36% | 42.49% |
| P2-07 | 29.47% | 48.78% | 28.94% | 24.16% | 58.81% | 74.52% | 76.69% | 79.83% | 74.76% | 36.29% |
| P2-08 | 30.72% | 53.65% | 38.67% | 26.35% | 53.07% | 77.24% | 78.60% | 81.93% | 79.57% | 32.58% |
| P2-09 | 32.88% | 40.26% | 38.44% | 24.01% | 65.02% | 3.96% | 80.08% | 83.28% | 79.16% | 42.19% |
| P2-10 | 62.58% | 70.73% | 51.99% | 31.49% | 56.32% | 4.16% | 73.92% | 82.46% | 64.37% | 48.35% |

It can be seen from FIG. 3 and Table 24 that the proportion of specific library molecules obtained by amplification using the combination of primers with phosphorothioate modification is significantly higher than that of the combination with no phosphorothioate modification.

### Embodiment 2

### Effect of Number of Phosphorothioates on Library Construction by Multiplex Linear Amplification

Free human plasma DNA samples were used for library construction by multiplexed linear amplifications using phosphorothioate-primers with different numbers of phosphorothioates. The success rate and specificity of library construction using the phosphorothioate-primers with different number of phosphorothioate were compared for screening suitable phosphorothioate-primers for target library construction with high specificity.

### Experimental Materials

### 1. Test Samples

The sample is a free human plasma DNA sample.

The sample was quantified using Qubit, and the concentration of the sample was set as 20 ng/μL. The test was repeated 6 times.

### 2. The Primer Sequences are Shown in Table 25. The Suppliers are all Sangon Biotech (Shanghai) Co., Ltd.

TABLE 25

| | | Concentration | |
|---|---|---|---|
| No. | Primer Name | (nM) | Primer Sequence |
| 1 | ALK_f19-12N | 100 | SEQ ID NO: 1 |
| 2 | ALK_f19-4N | 50 | SEQ ID NO: 2 |
| 3 | ALK_f19-n4N | 100 | SEQ ID NO: 3 |
| 6 | EGFR_i21-2N | 100 | SEQ ID NO: 6 |
| 10 | TP53_i07-2N | 100 | SEQ ID NO: 10 |

* The primers and sequences used in panel 3, panel 4, panel 5, panel 6, panel 7, and panel 8 are exactly the same, except that the primer for panel 4 does not have phosphorothioate modification, while the primers for panel 3, panel 5, panel 6, panel 7, and panel 8 have phosphorothioate modifications at the last 3, 1, 5, 8, and 12 phosphodiester bonds of the nucleotide backbones at the 3′ end. The sequence in bold represents the first universal sequence, and the sequence in non-bold represents the sequence complementary to the target region. All primers are modified with DL1 group.

### 3. The Single-Chain Adapters and Primers for Pre-Amplification.

The single-chain adapter and the primer for pre-amplification are the aforementioned UA4/UA5 and i3/i4 (the single-chain adapter is added according to Table 28).

The remaining experimental materials and equipments are the same as in those in Embodiment 1, and various experimental procedures are also similar with those of Embodiment 1.

### Experimental Results

1. The results of quality control of library after library construction by linear amplification with different combinations of primers:

TABLE 26

| | | Number of | Number of | Number of | Proportion |
|---|---|---|---|---|---|
| | | Initial Quality | Quality Control | Total | of quality |
| Sample | Primer | Control | Library | Library | control library |
| No. | Combination | Molecules | Molecules | Molecules | molecules |
| P3-01 | panel 3 | 10,000 | 15418 | 1.39E+05 | 11.08% |
| P3-02 | panel 3 | 10,000 | 15479 | 1.73E+05 | 8.95% |
| P3-03 | panel 3 | 10,000 | 16397 | 1.72E+05 | 9.54% |
| P3-04 | panel 3 | 10,000 | 16937 | 1.84E+05 | 9.22% |
| P3-05 | panel 3 | 10,000 | 19447 | 2.28E+05 | 8.53% |
| P3-06 | panel 3 | 10,000 | 16418 | 1.97E+05 | 8.35% |
| P4-01 | panel 4 | 10,000 | 8171 | 1.38E+06 | 0.59% |
| P4-02 | panel 4 | 10,000 | 7710 | 2.38E+06 | 0.32% |
| P4-03 | panel 4 | 10,000 | 7040 | 5.98E+05 | 1.18% |
| P4-04 | panel 4 | 10,000 | 8520 | 1.23E+06 | 0.69% |
| P4-05 | panel 4 | 10,000 | 9938 | 9.22E+06 | 0.11% |
| P4-06 | panel 4 | 10,000 | 7503 | 9.04E+05 | 0.83% |
| P5-01 | panel 5 | 10,000 | 10847 | 4.18E+05 | 2.60% |
| P5-02 | panel 5 | 10,000 | 13828 | 3.38E+05 | 4.09% |
| P5-03 | panel 5 | 10,000 | 14138 | 5.27E+05 | 2.68% |
| P5-04 | panel 5 | 10,000 | 14594 | 5.30E+05 | 2.75% |
| P5-05 | panel 5 | 10,000 | 11036 | 6.64E+05 | 1.66% |
| P5-06 | panel 5 | 10,000 | 11169 | 3.08E+05 | 3.62% |
| P6-01 | panel 6 | 10,000 | 17706 | 1.64E+05 | 10.77% |
| P6-02 | panel 6 | 10,000 | 13766 | 1.21E+05 | 11.40% |
| P6-03 | panel 6 | 10,000 | 16750 | 1.88E+05 | 8.92% |
| P6-04 | panel 6 | 10,000 | 15905 | 1.34E+05 | 11.86% |
| P6-05 | panel 6 | 10,000 | 12181 | 1.36E+05 | 8.97% |
| P6-06 | panel 6 | 10,000 | 15020 | 1.43E+05 | 10.49% |
| P7-01 | panel 7 | 10,000 | 6548 | 7.11E+04 | 9.21% |
| P7-02 | panel 7 | 10,000 | 5770 | 6.27E+04 | 9.19% |
| P7-03 | panel 7 | 10,000 | 6537 | 6.02E+04 | 10.86% |
| P7-04 | panel 7 | 10,000 | 8638 | 7.27E+04 | 11.88% |
| P7-05 | panel 7 | 10,000 | 7548 | 8.49E+04 | 8.89% |
| P7-06 | panel 7 | 10,000 | 5812 | 6.55E+04 | 8.87% |
| P8-01 | panel 8 | 10,000 | 5555 | 1.18E+05 | 3.27% |
| P8-02 | panel 8 | 10,000 | 3667 | 8.36E+04 | 2.79% |
| P8-03 | panel 8 | 10,000 | 2064 | 7.80E+04 | 2.18% |
| P8-04 | panel 8 | 10,000 | 4061 | 1.02E+05 | 3.82% |
| P8-05 | panel 8 | 10,000 | 2183 | 6.35E+04 | 4.31% |
| P8-06 | panel 8 | 10,000 | 2170 | 4.79E+04 | 3.03% |

It can be seen from the data in the above table that the performance of multiplexed linear amplification is associated with the number of phosphorothioate-modifications on the primers. When the number of phosphorothioate modifications is from 3 to 8 (panel 3, 6, 7), the library has good specificity and high conversion; and when the number of phosphorothioate modifications is less than 3 (panel 4, 5) or greater than 8 (panel 8), the library has poor specificity. At the same time, when the number of phosphorothioate modifications reaches 12 (panel 8), the conversion of the library significantly decreases.

2. Quantification of Number of Total Library Molecules After Library Amplification:

TABLE 27

| | Number | Number of | | | | |
|---|---|---|---|---|---|---|
| | of library | total library | | | | |
| | molecules | molecules | | Sample | Loading | Loading |
| Sample | after | after library | Amplification | Copy | Copy | Volume |
| No. | ligation | amplification | Multiple | Number/μL | Number | μL |
| P3-01 | 1.39E+05 | 1.83E+09 | 1.32E+04 | 9.16E+07 | 5.00E+08 | 5.46 |
| P3-02 | 1.73E+05 | 2.07E+09 | 1.20E+04 | 1.04E+08 | 5.00E+08 | 4.83 |
| P3-03 | 1.72E+05 | 2.30E+09 | 1.34E+04 | 1.15E+08 | 5.00E+08 | 4.34 |
| P3-04 | 1.84E+05 | 2.60E+09 | 1.42E+04 | 1.30E+08 | 5.00E+08 | 3.84 |
| P3-05 | 2.28E+05 | 3.23E+09 | 1.42E+04 | 1.62E+08 | 5.00E+08 | 3.09 |
| P3-06 | 1.97E+05 | 2.52E+09 | 1.28E+04 | 1.26E+08 | 5.00E+08 | 3.97 |
| P4-01 | 1.38E+06 | 1.12E+10 | 8.11E+03 | 5.59E+08 | 5.00E+08 | 0.89 |
| P4-02 | 2.38E+06 | 1.34E+10 | 5.64E+03 | 6.71E+08 | 5.00E+08 | 0.75 |
| P4-03 | 5.98E+05 | 6.30E+09 | 1.05E+04 | 3.15E+08 | 5.00E+08 | 1.59 |
| P4-04 | 1.23E+06 | 1.09E+10 | 8.91E+03 | 5.47E+08 | 5.00E+08 | 0.91 |
| P4-05 | 9.22E+06 | 1.86E+10 | 2.02E+03 | 9.32E+08 | 5.00E+08 | 0.54 |
| P4-06 | 9.04E+05 | 4.63E+09 | 5.12E+03 | 2.31E+08 | 5.00E+08 | 2.16 |
| P5-01 | 4.18E+05 | 5.91E+09 | 1.41E+04 | 2.95E+08 | 5.00E+08 | 1.69 |
| P5-02 | 3.38E+05 | 4.89E+09 | 1.44E+04 | 2.44E+08 | 5.00E+08 | 2.05 |
| P5-03 | 5.27E+05 | 7.46E+09 | 1.42E+04 | 3.73E+08 | 5.00E+08 | 1.34 |
| P5-04 | 5.30E+05 | 6.67E+09 | 1.26E+04 | 3.33E+08 | 5.00E+08 | 1.50 |
| P5-05 | 6.64E+05 | 9.47E+09 | 1.43E+04 | 4.74E+08 | 5.00E+08 | 1.06 |
| P5-06 | 3.08E+05 | 3.64E+09 | 1.18E+04 | 1.82E+08 | 5.00E+08 | 2.75 |
| P6-01 | 1.64E+05 | 2.03E+09 | 1.24E+04 | 1.02E+08 | 5.00E+08 | 4.92 |
| P6-02 | 1.21E+05 | 1.66E+09 | 1.38E+04 | 8.31E+07 | 5.00E+08 | 6.02 |
| P6-03 | 1.88E+05 | 2.01E+09 | 1.07E+04 | 1.01E+08 | 5.00E+08 | 4.97 |
| P6-04 | 1.34E+05 | 1.69E+09 | 1.26E+04 | 8.43E+07 | 5.00E+08 | 5.93 |
| P6-05 | 1.36E+05 | 1.79E+09 | 1.32E+04 | 8.95E+07 | 5.00E+08 | 5.58 |
| P6-06 | 1.43E+05 | 2.03E+09 | 1.42E+04 | 1.01E+08 | 5.00E+08 | 4.94 |
| P7-01 | 7.11E+04 | 7.25E+08 | 1.02E+04 | 3.62E+07 | 5.00E+08 | 13.80 |
| P7-02 | 6.27E+04 | 8.77E+08 | 1.40E+04 | 4.39E+07 | 5.00E+08 | 11.40 |
| P7-03 | 6.02E+04 | 7.81E+08 | 1.30E+04 | 3.91E+07 | 5.00E+08 | 12.80 |
| P7-04 | 7.27E+04 | 1.01E+09 | 1.39E+04 | 5.05E+07 | 5.00E+08 | 9.90 |
| P7-05 | 8.49E+04 | 1.00E+09 | 1.18E+04 | 5.01E+07 | 5.00E+08 | 9.97 |
| P7-06 | 6.55E+04 | 7.37E+08 | 1.12E+04 | 3.68E+07 | 5.00E+08 | 13.58 |
| P8-01 | 1.18E+05 | 1.72E+09 | 1.46E+04 | 8.59E+07 | 5.00E+08 | 5.82 |
| P8-02 | 8.36E+04 | 1.02E+09 | 1.22E+04 | 5.09E+07 | 5.00E+08 | 9.82 |
| P8-03 | 7.80E+04 | 8.21E+08 | 1.05E+04 | 4.11E+07 | 5.00E+08 | 12.17 |
| P8-04 | 1.02E+05 | 1.05E+09 | 1.02E+04 | 5.23E+07 | 5.00E+08 | 9.56 |
| P8-05 | 6.35E+04 | 9.16E+08 | 1.44E+04 | 4.58E+07 | 5.00E+08 | 10.91 |
| P8-06 | 4.79E+04 | 6.61E+08 | 1.38E+04 | 3.30E+07 | 5.00E+08 | 15.13 |

It can be seen from the data in the above table that the amplification efficiency of all the libraries meets the expectations.

3. Yield and quality of raw data:

TABLE 28

| Sample | Q30 | Single-Chain | Primers For Library Pre- |
|---|---|---|---|
| No. | (%) | Adapters | Amplification |
| P3-01 | 88.10 | UA4 | i3 |
| P3-02 | 83.51 | UA5 | i4 |
| P3-03 | 82.59 | UA4 | i3 |
| P3-04 | 84.55 | UA5 | i4 |
| P3-05 | 84.60 | UA4 | i3 |
| P3-06 | 85.89 | UA5 | i4 |
| P4-01 | 89.93 | UA4 | i3 |
| P4-02 | 81.93 | UA5 | i4 |
| P4-03 | 85.13 | UA4 | i3 |
| P4-04 | 84.43 | UA5 | i4 |
| P4-05 | 87.06 | UA4 | i3 |
| P4-06 | 83.46 | UA5 | i4 |
| P5-01 | 85.07 | UA4 | i3 |
| P5-02 | 84.29 | UA5 | i4 |
| P5-03 | 85.73 | UA4 | i3 |
| P5-04 | 84.68 | UA5 | i4 |
| P5-05 | 89.41 | UA4 | i3 |
| P5-06 | 80.97 | UA5 | i4 |
| P6-01 | 80.39 | UA4 | i3 |
| P6-02 | 84.38 | UA5 | i4 |
| P6-03 | 82.42 | UA4 | i3 |
| P6-04 | 89.64 | UA5 | i4 |
| P6-05 | 82.23 | UA4 | i3 |
| P6-06 | 80.18 | UA5 | i4 |
| P7-01 | 81.59 | UA4 | i3 |
| P7-02 | 85.33 | UA5 | i4 |
| P7-03 | 81.49 | UA4 | i3 |
| P7-04 | 86.31 | UA5 | i4 |
| P7-05 | 87.46 | UA4 | i3 |
| P7-06 | 87.92 | UA5 | i4 |
| P8-01 | 86.25 | UA4 | i3 |
| P8-02 | 85.83 | UA5 | i4 |
| P8-03 | 81.67 | UA4 | i3 |
| P8-04 | 88.44 | UA5 | i4 |
| P8-05 | 80.43 | UA4 | i3 |
| P8-06 | 84.53 | UA5 | i4 |

It can be seen from the data in the above table that the sequenced data quality of all the libraries meets the requirements.

4. Reads and proportions obtained by library construction from the linear amplification with different primers

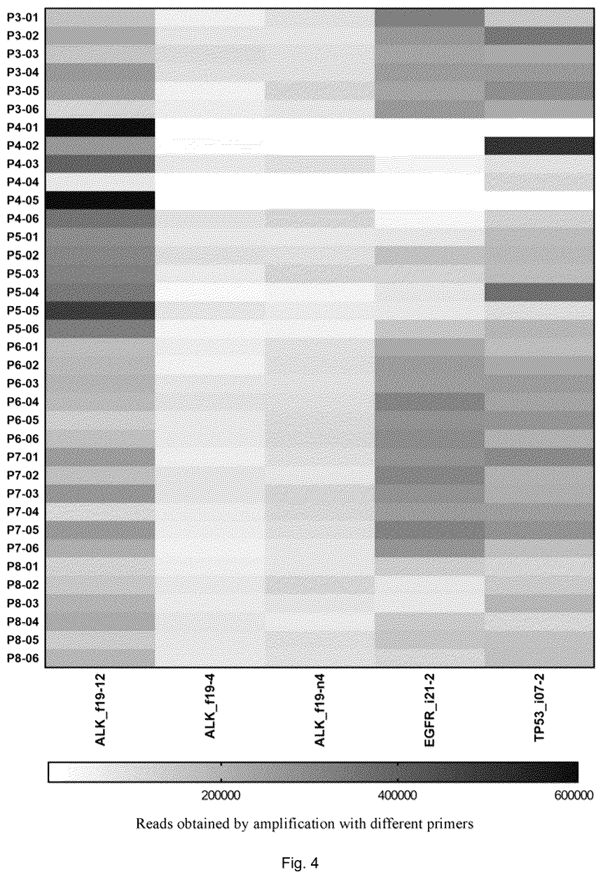

4.1. The number of reads obtained by library construction from the linear amplification with different primers. The results are shown in FIG. 4 and Table 29.

TABLE 29

| Sample | | ALK_{—} | ALK_{—} | ALK_{—} | EGFR_{—} | TP53_{—} |
|---|---|---|---|---|---|---|
| No. | Total | f19-12 | f19-4 | f19-n4 | i21-2 | i07-2 |
| P3-01 | 797683 | 164564 | 59599 | 95161 | 326509 | 151850 |
| P3-02 | 1007044 | 223602 | 83915 | 83159 | 267233 | 349135 |
| P3-03 | 816277 | 157037 | 105213 | 94675 | 235397 | 223955 |
| P3-04 | 968080 | 263864 | 95506 | 93508 | 253725 | 261477 |
| P3-05 | 945432 | 249282 | 55901 | 116089 | 235236 | 288924 |
| P3-06 | 754998 | 123415 | 64816 | 80528 | 263815 | 222424 |
| P4-01 | 639037 | 598909 | 9790 | 4280 | 20947 | 5111 |
| P4-02 | 832211 | 268132 | 26545 | 22348 | 9517 | 505669 |
| P4-03 | 694988 | 395368 | 70101 | 93989 | 45708 | 89822 |
| P4-04 | 252302 | 71283 | 17994 | 23225 | 21791 | 118009 |
| P4-05 | 640702 | 603768 | 5912 | 22773 | 4144 | 4105 |
| P4-06 | 725698 | 352411 | 89522 | 116755 | 39710 | 127300 |
| P5-01 | 698410 | 292598 | 76517 | 70865 | 79004 | 179426 |
| P5-02 | 850890 | 308726 | 96662 | 95029 | 165848 | 184625 |
| P5-03 | 810618 | 320381 | 67605 | 119802 | 122385 | 180445 |
| P5-04 | 878036 | 346235 | 33040 | 48085 | 83094 | 367582 |
| P5-05 | 813157 | 489118 | 82028 | 68600 | 71469 | 101942 |
| P5-06 | 772789 | 332328 | 48503 | 51391 | 147825 | 192742 |
| P6-01 | 735272 | 177859 | 62953 | 94875 | 223653 | 175932 |
| P6-02 | 812742 | 187118 | 55446 | 96705 | 253830 | 219643 |
| P6-03 | 875665 | 196616 | 81268 | 90627 | 251445 | 255709 |
| P6-04 | 920662 | 180975 | 89664 | 98902 | 314007 | 237114 |
| P6-05 | 872204 | 138795 | 70217 | 107529 | 282606 | 273057 |
| P6-06 | 855094 | 172882 | 70731 | 111615 | 293565 | 206301 |
| P7-01 | 997459 | 256305 | 62517 | 96127 | 282357 | 300153 |
| P7-02 | 851328 | 171237 | 84081 | 80445 | 310480 | 205085 |
| P7-03 | 971890 | 270653 | 85072 | 122853 | 280687 | 212625 |
| P7-04 | 793155 | 113284 | 66184 | 109753 | 255100 | 248834 |
| P7-05 | 1021896 | 264933 | 64222 | 96402 | 317142 | 279197 |
| P7-06 | 803162 | 214155 | 57192 | 79460 | 276621 | 175734 |
| P8-01 | 529794 | 126531 | 61966 | 82283 | 136752 | 122262 |
| P8-02 | 572907 | 150872 | 76799 | 118885 | 83138 | 143213 |
| P8-03 | 619986 | 203746 | 66488 | 85904 | 76191 | 187657 |
| P8-04 | 634987 | 218043 | 83563 | 68107 | 143855 | 121419 |
| P8-05 | 641554 | 140549 | 78192 | 102200 | 155334 | 165279 |
| P8-06 | 637048 | 190208 | 71224 | 91277 | 117682 | 166657 |

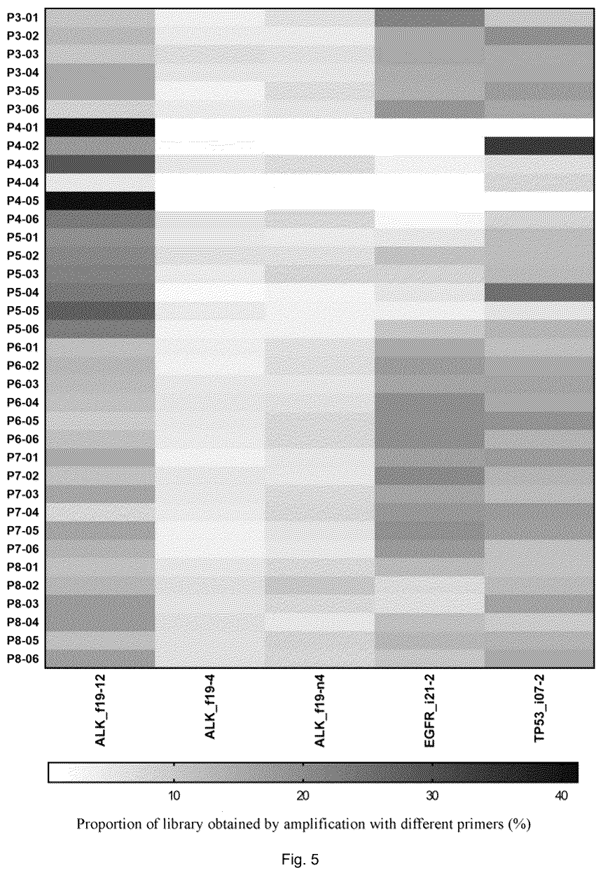

4.2. The results of the proportion of reads obtained by library construction from the linear amplification with different primers are shown in FIG. 5 and Table 30.

TABLE 30

| Sample No. | ALK_f19-12 | ALK_f19-4 | ALK_f19-n4 | EGFR_i21-2 | TP53_i07-2 |
|---|---|---|---|---|---|
| P3-01 | 20.63% | 7.47% | 11.93% | 40.93% | 19.04% |
| P3-02 | 22.20% | 8.33% | 8.26% | 26.54% | 34.67% |
| P3-03 | 19.24% | 12.89% | 11.60% | 28.84% | 27.44% |
| P3-04 | 27.26% | 9.87% | 9.66% | 26.21% | 27.01% |
| P3-05 | 26.37% | 5.91% | 12.28% | 24.88% | 30.56% |
| P3-06 | 16.35% | 8.58% | 10.67% | 34.94% | 29.46% |
| P4-01 | 93.72% | 1.53% | 0.67% | 3.28% | 0.80% |
| P4-02 | 32.22% | 3.19% | 2.69% | 1.14% | 60.76% |
| P4-03 | 56.89% | 10.09% | 13.52% | 6.58% | 12.92% |
| P4-04 | 28.25% | 7.13% | 9.21% | 8.64% | 46.77% |
| P4-05 | 94.24% | 0.92% | 3.55% | 0.65% | 0.64% |
| P4-06 | 48.56% | 12.34% | 16.09% | 5.47% | 17.54% |
| P5-01 | 41.89% | 10.96% | 10.15% | 11.31% | 25.69% |
| P5-02 | 36.28% | 11.36% | 11.17% | 19.49% | 21.70% |
| P5-03 | 39.52% | 8.34% | 14.78% | 15.10% | 22.26% |
| P5-04 | 39.43% | 3.76% | 5.48% | 9.46% | 41.86% |
| P5-05 | 60.15% | 10.09% | 8.44% | 8.79% | 12.54% |
| P5-06 | 43.00% | 6.28% | 6.65% | 19.13% | 24.94% |
| P6-01 | 24.19% | 8.56% | 12.90% | 30.42% | 23.93% |
| P6-02 | 23.02% | 6.82% | 11.90% | 31.23% | 27.02% |
| P6-03 | 22.45% | 9.28% | 10.35% | 28.71% | 29.20% |
| P6-04 | 19.66% | 9.74% | 10.74% | 34.11% | 25.75% |
| P6-05 | 15.91% | 8.05% | 12.33% | 32.40% | 31.31% |
| P6-06 | 20.22% | 8.27% | 13.05% | 34.33% | 24.13% |
| P7-01 | 25.70% | 6.27% | 9.64% | 28.31% | 30.09% |
| P7-02 | 20.11% | 9.88% | 9.45% | 36.47% | 24.09% |
| P7-03 | 27.85% | 8.75% | 12.64% | 28.88% | 21.88% |
| P7-04 | 14.28% | 8.34% | 13.84% | 32.16% | 31.37% |
| P7-05 | 25.93% | 6.28% | 9.43% | 31.03% | 27.32% |
| P7-06 | 26.66% | 7.12% | 9.89% | 34.44% | 21.88% |
| P8-01 | 23.88% | 11.70% | 15.53% | 25.81% | 23.08% |
| P8-02 | 26.33% | 13.41% | 20.75% | 14.51% | 25.00% |
| P8-03 | 32.86% | 10.72% | 13.86% | 12.29% | 30.27% |
| P8-04 | 34.34% | 13.16% | 10.73% | 22.65% | 19.12% |
| P8-05 | 21.91% | 12.19% | 15.93% | 24.21% | 25.76% |
| P8-06 | 29.86% | 11.18% | 14.33% | 18.47% | 26.16% |

It can be seen from FIG. 4 to FIG. 5 and Table 30 to Table 31 that the uniformity of the amplification of the system will increase with the increased number of phosphorothioate modifications. When the number of phosphorothioate modifications exceeds 3, the multiplexed linear amplification system has good uniformity, and the probability of non-specific PCR amplification is greatly reduced from 67% to 0%.

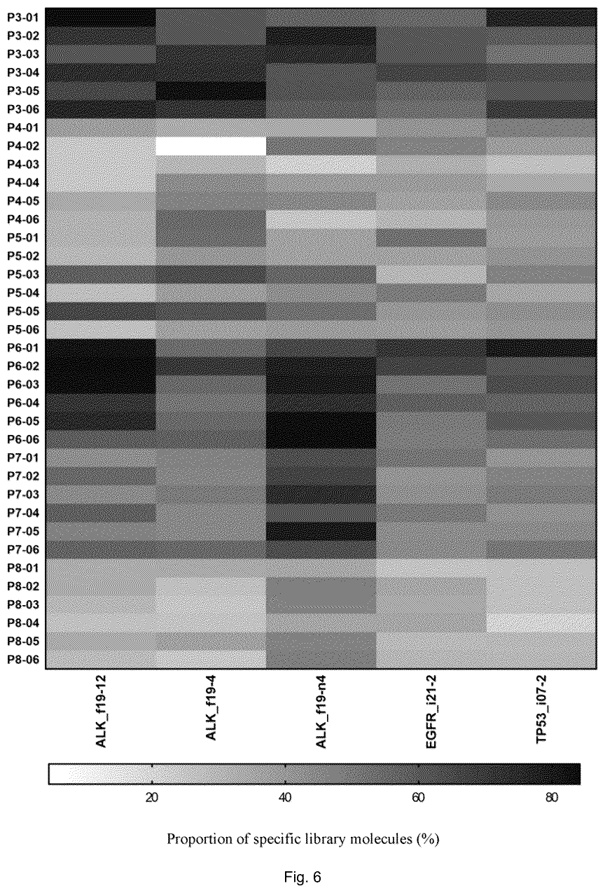

5. The results of proportion of specificity of library molecules obtained by the linear amplification with different primers are shown in FIG. 6 and Table 31. The proportion of specificity is calculated by dividing the number of ontarget reads for each primer by the number of reads for each primer (that is, the number of reads obtained by library construction from the linear amplification with the respective primers in Table 29)×100%.

TABLE 31

| Sample No. | ALK_f19-12 | ALK_f19-4 | ALK_f19-n4 | EGFR_i21-2 | TP53_i07-2 |
|---|---|---|---|---|---|
| P3-01 | 83.92% | 59.95% | 56.78% | 55.06% | 77.10% |
| P3-02 | 71.80% | 60.35% | 78.18% | 60.03% | 59.16% |
| P3-03 | 60.92% | 72.22% | 74.44% | 61.13% | 51.74% |
| P3-04 | 74.10% | 72.91% | 61.50% | 66.60% | 63.72% |
| P3-05 | 65.61% | 82.90% | 61.78% | 56.80% | 60.43% |
| P3-06 | 76.24% | 71.06% | 58.89% | 53.46% | 69.36% |
| P4-01 | 38.17% | 32.72% | 33.23% | 41.58% | 47.95% |
| P4-02 | 24.08% | 4.51% | 50.45% | 46.76% | 38.35% |
| P4-03 | 23.80% | 29.63% | 21.62% | 32.10% | 26.89% |
| P4-04 | 23.82% | 43.54% | 38.62% | 38.71% | 33.10% |
| P4-05 | 34.05% | 46.11% | 44.88% | 36.51% | 43.68% |
| P4-06 | 31.28% | 54.35% | 24.89% | 30.05% | 39.48% |
| P5-01 | 31.37% | 53.70% | 36.46% | 52.15% | 38.38% |
| P5-02 | 29.34% | 40.10% | 36.58% | 36.52% | 41.05% |
| P5-03 | 57.93% | 63.83% | 55.63% | 29.51% | 46.71% |
| P5-04 | 27.42% | 37.68% | 42.79% | 49.01% | 34.50% |
| P5-05 | 65.84% | 62.20% | 52.72% | 39.86% | 42.36% |
| P5-06 | 26.95% | 36.82% | 38.86% | 38.10% | 40.27% |
| P6-01 | 82.01% | 54.13% | 65.71% | 70.88% | 79.77% |
| P6-02 | 83.92% | 70.15% | 78.37% | 66.96% | 61.43% |
| P6-03 | 83.57% | 54.78% | 80.26% | 51.08% | 63.60% |
| P6-04 | 71.50% | 50.85% | 73.28% | 58.03% | 56.65% |
| P6-05 | 75.34% | 54.74% | 83.39% | 49.38% | 60.06% |
| P6-06 | 58.91% | 57.56% | 84.16% | 48.82% | 53.21% |
| P7-01 | 42.98% | 46.94% | 63.89% | 50.99% | 40.14% |
| P7-02 | 54.86% | 45.65% | 66.91% | 41.52% | 46.94% |
| P7-03 | 43.74% | 48.96% | 74.39% | 42.32% | 49.09% |
| P7-04 | 57.68% | 44.96% | 61.08% | 49.31% | 41.89% |
| P7-05 | 46.37% | 44.74% | 80.37% | 43.42% | 43.56% |
| P7-06 | 56.64% | 55.01% | 63.87% | 43.67% | 50.43% |
| P8-01 | 32.88% | 33.92% | 34.83% | 25.68% | 26.80% |
| P8-02 | 33.40% | 27.31% | 47.91% | 35.43% | 27.10% |
| P8-03 | 29.81% | 24.87% | 46.66% | 34.12% | 26.73% |
| P8-04 | 26.55% | 25.49% | 35.51% | 35.72% | 20.52% |
| P8-05 | 33.80% | 36.83% | 46.84% | 28.86% | 28.89% |
| P8-06 | 28.49% | 24.19% | 48.22% | 30.65% | 29.78% |

It can be seen from FIG. 6 and Table 31 that the specificity of multiplexed linear amplification is associated with the number of phosphorothioate modifications on the primers. When the number of phosphorothioate-modifications is from 3 to 8, the library has good specificity; and when the number of phosphorothioate-modifications is less than 3 or greater than 8, the library has poor specificity.

### Embodiment 3

### Effects of Different Duality Functional Groups of Primers on Specificity of Library Construction by Linear Amplification

Linear amplifications were carried out with phosphorothioate-primers modified by different duality functional groups. Library was constructed using free human plasma DNA samples. The specificities of library construction using the phosphorothioate-primers with different duality functional groups were compared for screening suitable phosphorothioate-primers for target library construction with high specificity.

### Experimental Materials

### 1. Test Samples

The sample is a free human plasma DNA sample.

The sample was quantified by qPCR, and the cfDNA quantification system and qPCR program were shown in Table 7 and Table 8 above.

### 2. Primer Sequences (the Suppliers are all Sangon Biotech (Shanghai) Co., Ltd.)

The sequences of the specific primers (P01 to P34) used in Embodiment 3 are exactly the same as the EGFR_i21-2N (SEQ ID NO: 6), expect for different modifications as made. The specific modifications are described below: The primers P01 and P02 do not have duality functional groups, and the number of phosphorothioate modifications on the last nucleotides at the 3′ end is 0 and 3, respectively; the duality functional group of the primers P03, P04 and P05 is NH2-C6 group, and the number of phosphorothioate modifications on the last nucleotides at the 3′ end is 0, 5 and 3, respectively; the duality functional group of the primers P06, P07 and P08 is DL1, and the number of phosphorothioate modifications on the last nucleotides at the 3′ end is 0, 3 and 5, respectively; the duality functional group of the primer P09 is DL2 (LNA modification), and the number of phosphorothioate modifications on the last nucleotides at the 3′ end is 3; the duality functional groups of the primers P10-P23 are all 3, while the duality functional group of P10 is DL3, the duality functional group of P11 is DL4, the duality functional group of P12 is DL5, the duality functional group of P13 is a C6 Spacer, the duality functional group of P14 is DL6, the duality functional group of P15 is an Invert T, the duality functional group of P16 is DL7, the duality functional group of P17 is a phosphate group, the duality functional group of P18 is DL8, the duality functional group of P19 is a C3 Spacer, the duality functional group of P20 is DL9, the duality functional group of P21 is SH—C6, the duality functional group of P22 is DL10; the duality functional group of the primers P23 and P24 is DL11, and the number of phosphorothioate modifications on the last nucleotides at the 3′ end is 3 and 0, respectively; the duality functional group of the primers P25 and P26 is DL12, and the number of phosphorothioate modifications on the last nucleotides at the 3′ end is 3 and 0, respectively; the duality functional group of the primers P27 and P28 is DL13, and the number of phosphorothioate modifications on the last nucleotides at the 3′ end is 3 and 0, respectively; the duality functional group of the primers P29 and P30 is DL14, and the number of phosphorothioate modifications on the last nucleotides at the 3′ end is 3 and 0, respectively; the duality functional group of the primers P31 and P32 is DL15, and the number of phosphorothioate modifications on the last nucleotides at the 3′ end is 3 and 0, respectively; and the duality functional group of the primers P33 and P34 is DL16, and the number of phosphorothioate modifications on the last nucleotides at the 3′ end is 3 and 0, respectively.

### 3. The Single-Chain Adapter and the Primer for Pre-Amplification are the Aforementioned UA4 and i3.

The remaining experimental materials and equipments are the same as those in Embodiment 1, and various experimental procedures are substantially by reference to Parts 1-3 of Embodiment 1. The number of fed sample DNA molecules is the number of molecules detected by the qPCR system in Table 7, that is, the amount of the initially fed DNA. Moreover, additional steps are as follows:

The amplification efficiency was detected after the linear amplification in step 1. 2 μL of the linear amplification product was taken, and 18 μL of TE buffer was added for 10× dilution. The linear amplification products were quantified with the qPCR detection system. The linear amplification multiple=the number of molecules of linear amplification products/the number of fed sample DNA molecules. *The number of molecules of the linear amplification product is the number of molecules detected by the qPCR system in Table 7.

The purification efficiency was detected after purification of the linear amplification productions in step 2. 2 μL of the purified linear amplification product was taken, and 18 μL of TE buffer was added for 10× dilution. The purified linear amplification products were quantified with the qPCR detection system. The linear amplification multiple after purification=the number of molecules of the purified linear amplification products/the number of fed sample DNA molecules. *The purification efficiency=the number of molecules of purified linear amplification products/the number of molecules of linear amplification products. *The number of molecules of the purified linear amplification product is the number of molecules detected by the qPCR system in Table 7.

The Experimental Results are in Particular Shown in the Table Below, where:

Ligation efficiency=the number of specific library molecules/the number of molecules of the purified linear amplification product

Conversion rate of the specific library molecules=the number of specific library molecules/the number of fed sample DNA molecules

Proportion of specific library molecules=the number of specific library molecules/the number of total library molecules

*The number of specific library molecules is the number of molecules detected with the qPCR system in Table 13. *The number of total library molecules is the number of molecules detected with the qPCR system in Table 18.

TABLE 32

| | Number | | | | | | |
|---|---|---|---|---|---|---|---|
| | of Fed | | | | | Conversion | |
| | Sample | | | | | on rate of | Proportion |
| | DNA | Linear | Amplification | | | specific | of Specific |
| Sample | Molecule | amplification | multiple after | Purification | Ligation | library | Library |
| No. | (Copies) | multiple | purification | efficiency | efficiency | molecules | Molecules |
| P01 | 6000 | 30.3 | 10.2 | 0.34 | 1% | 0.12 | 0.0% |
| P02 | 6000 | 23.2 | 8.6 | 0.37 | 10% | 0.89 | 1.0% |
| P03 | 6000 | 16.5 | 4.2 | 0.25 | 32% | 1.34 | 6.20% |
| P04 | 6000 | 10.8 | 2.9 | 0.27 | 31% | 0.90 | 4% |
| P05 | 6000 | 12.1 | 3.5 | 0.29 | 30% | 1.05 | 5.8% |
| P06 | 6000 | 28.4 | 9.3 | 0.33 | 31% | 2.88 | 14.20% |
| P07 | 6000 | 25.2 | 8.2 | 0.33 | 32% | 2.62 | 25.60% |
| P08 | 6000 | 15.1 | 5.4 | 0.36 | 29% | 1.57 | 30.6 |
| P09 | 6000 | 35.5 | 7.4 | 0.21 | 30% | 2.22 | 2.0% |
| P10 | 6000 | 14.4 | 3.7 | 0.26 | 42% | 1.55 | 27.9% |
| P11 | 6000 | 20.8 | 7.5 | 0.36 | 30% | 2.25 | 10.8% |
| P12 | 6000 | 15.6 | 5.2 | 0.33 | 32% | 1.66 | 8.5% |
| P13 | 6000 | 4.8 | 1.6 | 0.33 | 25% | 0.40 | 15.0% |
| P14 | 6000 | 20.3 | 6.5 | 0.32 | 28% | 1.82 | 25% |
| P15 | 6000 | 4.5 | 1.1 | 0.24 | 23% | 0.25 | 0.7% |
| P16 | 6000 | 6.2 | 1.5 | 0.24 | 30% | 0.45 | 1.4% |
| P17 | 6000 | 4.8 | 1.3 | 0.27 | 27% | 0.35 | 2.1% |
| P18 | 6000 | 18.8 | 6.2 | 0.33 | 27% | 1.66 | 7.8% |
| P19 | 6000 | 16.4 | 5.4 | 0.33 | 25% | 1.35 | 6.8% |
| P20 | 6000 | 25.6 | 8.9 | 0.35 | 30% | 2.67 | 35.1% |
| P21 | 6000 | 15.8 | 6.4 | 0.41 | 28% | 1.79 | 12.3% |
| P22 | 6000 | 26.4 | 8.5 | 0.32 | 32% | 2.72 | 15.6% |
| P23 | 6000 | 8.2 | 2.2 | 0.27 | 31% | 0.68 | 3.8% |
| P24 | 6000 | 10.2 | 3.2 | 0.31 | 31% | 0.99 | 4.5% |
| P25 | 6000 | 28.3 | 7.9 | 0.28 | 27% | 2.13 | 24.9% |
| P26 | 6000 | 30.3 | 10.2 | 0.34 | 27% | 2.75 | 15.9% |
| P27 | 6000 | 24.2 | 7.7 | 0.32 | 26% | 2.00 | 25.0% |
| P28 | 6000 | 25.2 | 6.7 | 0.27 | 26% | 1.74 | 20.4% |
| P29 | 6000 | 25.7 | 6.7 | 0.26 | 22% | 1.47 | 12.8% |
| P30 | 6000 | 28.7 | 8.7 | 0.30 | 32% | 2.78 | 22.8% |
| P31 | 6000 | 25.2 | 6.8 | 0.27 | 34% | 2.31 | 17.6% |
| P32 | 6000 | 26.2 | 8.8 | 0.34 | 34% | 2.99 | 17.2% |
| P33 | 6000 | 20.4 | 6.3 | 0.31 | 33% | 2.08 | 18.2% |
| P34 | 6000 | 22.4 | 7.3 | 0.33 | 33% | 2.41 | 13.2% |

It can be seen from the above table that, compared with P01 and P02 which do not have duality functional group modification, the linear amplification with other primers modified by duality functional group has significantly higher ligation efficiency, conversion rate of specific library molecules and proportion of specific library molecules.

To sum up, the present disclosure effectively overcomes various shortcomings in the prior art and has high industrial application value.

The foregoing embodiments only illustrate the principles and effects of the present disclosure, but are not intended to limit the present disclosure. Any person skilled in the art can modify or change the above embodiments without departing from the spirit and scope of the present disclosure. Therefore, all equivalent modifications or changes made by persons skilled in the art without departing from the spirit and technical concepts disclosed in the present disclosure should still be encompassed by the appended claims of the present disclosure.

1. A method for amplifying a DNA target region, comprising: performing linear amplification of a fragmented DNA including the target region by a specific primer to provide a linear amplification product, wherein 3′ end of the specific primer is modified by a duality functional group, at least one phosphodiester bond of a part of nucleotide backbone at the 3′ end of the specific primer is modified by phosphorothioate, and the duality functional group is used to prevent the 3′ end of the specific primer from ligation with other oligonucleotides and can be removed by a specific enzyme to allow the specific primer to perform linear amplification.

2. The method for amplifying a DNA target region according to claim 1, wherein the linear amplification is a multiplexed amplification in which a number of target regions as targeted is ≥2;
and/or, the fragmented DNA has a length of 25 to 500 bp/nt, preferably 50 to 200 bp/nt;
and/or, the fragmented DNA has a structure of double-stranded DNA, single-stranded DNA or cDNA;
and/or, the fragmented DNA is free DNA;
and/or, the fragmented DNA originates from body fluid, preferably from blood and/or urine;
and/or, the fragmented DNA is prepared from a genomic DNA by fragmentation, preferably by ultrasonic fragmentation and/or enzymatic fragmentation.

3. The method for amplifying a DNA target region according to claim 1, wherein an amplification system for the linear amplification comprises the specific primer, a DNA polymerase and dNTPs, preferably, the DNA polymerase has 3′-5′ exonuclease activity, and more preferably, the DNA polymerase is selected from Family B DNA polymerases.

4. The method for amplifying a DNA target region according to claim 1, wherein an annealing temperature during the linear amplification ranges from 60° C. to 75° C., preferably from 65° C. to 72° C.

5. The method for amplifying a DNA target region according to claim 1, wherein at least a part of sequence at the 3′ end of the specific primer is complementary to the target region, and the part of sequence complementary to the target region has a length of ≥16 nt;
and/or, the specific primer further comprises one or a combination of a first universal sequence, a first sample index sequence, and a first sequencing sequence;
and/or, a nucleotide sequence of the specific primer comprises one or a combination of the sequences shown in SEQ ID NOs: 1-10.

6. The method for amplifying a DNA target region according to claim 1, wherein, in the specific primer, the number of phosphorothioate-modified nucleotides is 1 to 11, preferably 3 to 8;
and/or, in the specific primer, the phosphorothioate-modified nucleotides are contiguous;
and/or, in the specific primer, the number of the phosphorothioate-modified nucleotides located at the 3′ end of the specific primer is 1 to 11, preferably 3 to 8.

7. The method for amplifying a DNA target region according to claim 1, wherein a hydroxyl group attached to the 3-position carbon atom of a nucleotide at the 3′ end of the specific primer is replaced by a duality functional group, wherein the duality functional group at the 3′ end of the specific primer is selected from the group consisting of C3 Spacer group, Invert T group, phosphate group, biotin group, C6 Spacer group, NH₂—C6 group, and SH—C6 group;
and/or, a hydroxyl group attached to the 3-position carbon atom of a nucleotide at the 3′ end of the specific primer is replaced by a duality functional group, wherein the duality functional group at the 3′ end of the specific primer is a nucleotide complex group having a structure of:
wherein the Base is selected from the group consisting of base A, base G, base C, base T or base U;
R1 is selected from the group consisting of hydroxyl group, C3 Spacer group, Invert T group, phosphate group, biotin group, C6 Spacer group, NH₂—C6 group or SH—C6 group;
R2 is selected from the group consisting of hydrogen atom, fluorine atom, hydroxyl group, or methoxy group.

8. The method for amplifying a DNA target region according to claim 1, further comprising: purifying the linear amplification product, preferably, the purification method is affinity purification for labelled dNTP molecule;
and/or, at least part of the dNTPs are coupled with a label molecule, preferably, the label molecule is biotin.

9. A method for constructing a library, comprising: constructing the library by the linear amplification product provided in claim 1.

10. The method for constructing a library according to claim 9, comprising: ligating the linear amplification product to a single-stranded adapter by a single-stranded ligase to obtain a ligation product, wherein the single-chain adapter comprises one or a combination of a second sequencing sequence, a second sample index sequence, a second universal sequence, and a unique molecular index sequence.

11. The method for constructing a library according to claim 10, wherein the single-stranded ligase is a T4 RNA ligase or a thermostable RNA ligase;
and/or, a nucleotide at 5′ end of the single-chain adapter is modified, and the single-chain adapter has a single-stranded structure at a reaction temperature of adapter ligation, preferably, a phosphate group or an adenosine group is attached to a 5-position carbon atom of the nucleotide at the 5′ end of the single-stranded adapter,
and/or, a hydroxyl group attached to the 3-position carbon atom of a nucleotide at the 3′ end of the single-chain adapter is replaced by a blocking group, preferably, the blocking group at the 3′ end of the single-chain adapter is selected from the group consisting of Invert T group, phosphate group, biotin group, C6 Spacer group, NH₂—C6 group, SH—C6 group, and C3 Spacer group;
and/or, the 5′ end region of the single-chain adapter is a partially double-stranded structure with a sticky end;
and/or, a nucleotide sequence of the single-chain adapter comprises a sequence shown in one or more of SEQ ID NOs: 11-12.

12. The method for constructing a library according to claim 11, further comprising: pre-amplifying the ligation product to provide a pre-amplification product, wherein a forward primer of pre-amplification primers comprises one or a combination of a sequence complementary to the first universal sequence, a sequence complementary to the first sample index sequence, and a sequence complementary to the first sequencing sequence, and a reverse primer of the pre-amplification primers comprises a sequence complementary to the second sequencing sequence.

13. The method for constructing a library according to claim 12, further comprising: allowing the pre-amplification product to undergo library amplification to provide a library product, wherein a forward primer of library amplification primers comprises a sequence complementary to the first sequencing sequence, and a reverse primer of the library amplification primers comprises a sequence complementary to the second sequencing sequence.

14. A method for sequencing a DNA target region, comprising: sequencing the library product provided in claim 13 to provide a sequencing result of the target region.

15. A kit for amplifying a DNA target region, wherein the kit is suitable to the method for amplifying a DNA target region according to claim 1.

16. A kit for amplifying a DNA target region, wherein the kit is suitable to the method for constructing a library according to claim 9.

17. A kit for amplifying a DNA target region, wherein the kit is suitable to the method for sequencing a DNA target region according to claim 14.
